(19) United States
(12) Patent Application Publication
(10) Pub. No.: US 2014/0243387 A1
(43) Pub. Date: Aug. 28, 2014

(54) METHODS FOR IMPROVING CARDIAC CONTRACTILITY

(71) Applicants: Icahn School of Medicine at Mount Sinai, New York, NY (US); Sanford-Burnham Medical Research Institute, La Jolla, CA (US)

(72) Inventors: Mark Mercola, Rancho Santa Fe, CA (US); Agustin Rojas-Munoz, La Jolla, CA (US); Christine Wahlquist, San Diego, CA (US); Alexandre Colas, San Diego, CA (US); Roger J. Hajjar, Tenafly, NJ (US); Dongtak Jeong, Fort Lee, NJ (US)

(73) Assignees: Icahn School of Medicine at Mount Sinai, New York, NY (US); Sanford-Burnham Medical Research Institute, La Jolla, CA (US)

(21) Appl. No.: 14/109,460

(22) Filed: Dec. 17, 2013

Related U.S. Application Data
(60) Provisional application No. 61/738,963, filed on Dec. 18, 2012.

(51) Int. Cl. C12N 15/113; G01N 33/50

(52) U.S. Cl. CPC C12N 15/1137; G01N 33/5023
USPC 514/44 A; 435/6.13

(57) ABSTRACT

The present invention relates to regulation of cardiac contractile function. The present invention is based on the discovery that microRNAs contribute to the loss of cardiac contractility. Specifically, miR-25 binds to SERCA2a which results in a loss of function and interferes with Ca²⁺ handling. Accordingly, the present invention relates to methods of increasing cardiac contractile function by inhibiting miR-25. The invention further provides methods to identify agents that can modulate miR-25 activity, including high throughput screening methods, and provides a means to identify agents that are useful for treating patients having cardiac contractile function associated disorders.

### CROSS REFERENCE TO RELATED APPLICATIONS

This application claims the benefit of priority under 35 USC §119(e) to U.S. Application Ser. No. 61/738,963, filed Dec. 18, 2012. The disclosure of the prior application is considered part of and is incorporated by reference in its entirety in the disclosure of this application.

### GRANT INFORMATION

This invention was made with government support under Grant No. RC1-0000132 awarded by the California Institute for Regenerative Medicine; Grant Nos. NIH HL113601, HL108176, R01 HL093183, HL088434, P20HL100396 awarded by the National Institutes of Health; Contract No. HHSN268201000045C, and Grant No. P50 HL112324 awarded by National Heart, Lung, and Blood Institute Program of Excellence in Nanotechnology (PEN) Award. The government has certain rights in the invention.

### INCORPORATION OF SEQUENCE LISTING

The material in the accompanying Sequence Listing is hereby incorporated by reference into this application. The accompanying sequence listing text file, name BURN1640_{—}1_Sequence_Listing, was created on Dec. 16, 2013 and is 3 KB. The file can be assessed using Microsoft Word on a computer that uses Windows OS.

### BACKGROUND OF THE INVENTION

1. Field of the Invention

The invention relates generally to regulation of myocardial contraction and, more specifically, to improvement of cardiac contraction via inhibition of microRNAs.

2. Background Information

Heart failure is the culmination of diverse cardiovascular diseases, including hypertension, ischemic disease and atherosclerosis, valvular insufficiency, myocarditis, or contractile protein mutations. Despite varying etiologies and manifestations, heart failure is uniformly characterized by a progressive loss of contractility and heart function. The prevailing therapeutic strategy is to block the deleterious effects of the renin-angiotensin and sympathetic systems, but existing drugs target few mechanisms within the failing cardiomyocyte and there is a critical need for novel drugs especially in patients with advanced heart failure. A complex intracellular network balances contractility and Ca²⁺ handling in relationship to workload, and has been extensively modeled. Missing from current models is the role of microRNAs (miRs). miRs evolved to fine-tune nearly all normal and pathological processes examined by downregulating proteins that occupy key nodal points. It was reasoned that miRs that repress contractility might be upregulated during human heart failure, and might therefore constitute novel targets for therapeutic intervention.

The calcium-transporting ATPase SERCA2a, also known as ATP2A2, is the primary mechanism for Ca²⁺ uptake during excitation-contraction coupling in cardiomyocytes. Impaired Ca²⁺ uptake resulting from decreased expression and reduced activity of SERCA2a is a hallmark of heart failure. Accordingly, restoration of SERCA2a by gene transfer has proven effective in improving key parameters of heart failure in animal models and more recently in clinical trials. Reasoning that certain miRs that downregulate SERCA2 might be elevated in heart failure and compromise cardiac function, an assay was developed to functionally screen a whole-genome collection of miRs for selective downregulation of the Ca²⁺ pump.

MicroRNAs (miRs) are 18-24 nucleotides single-stranded RNAs associated with a protein complex called the RNA-induced silencing complex (RISC). Small RNAs are usually generated from non-coding regions of gene transcripts and function to suppress gene expression by translational repression and mRNA destabilization.

Heart failure is uniformly characterized by a progressive loss of contractile function. Whole genome screening of microRNAs (miRs) was used to discover that miR-25 is a natural negative regulator of cardiac contractility through its action on intracellular Ca²⁺ handling. Specific targets of miR-25 include the sarcoplasmic reticulum Ca²⁺ uptake pump SERCA2a. miR-25 is upregulated in human heart failure, and a specific anti-miR that blocks miR-25 function in vitro was able to restore SERCA2a levels following intravenous injection in a murine model of established heart failure, resulting in substantially improved contractile function and reduced myocardial fibrosis. These data point to therapeutic modulation of miR-25 as a potential means to treat heart failure.

### SUMMARY OF THE INVENTION

The present invention relates to regulation of cardiac contractile function. The present invention is based on the discovery that microRNAs contribute to the loss of cardiac contractility. Specifically, miR-25 binds to both SERCA2a which results in a loss of function and interferes with Ca²⁺ handling. Accordingly, the present invention relates to methods of increasing cardiac contractile function by inhibiting miR-25. The invention further provides methods to identify agents that can modulate miR-25 activity, including high throughput screening methods, and provides a means to identify agents that are useful for treating patients having cardiac contractile function associated disorders.

Accordingly, in one embodiment, the present invention provides a method of increasing contractility of heart muscle or cardiomyocytes in a subject comprising administering a miR-25 inhibitor. In one aspect, the miR-25 inhibitor is selected from the group consisting of an antagonist, a peptide, a polynucleotide, an antibody, a polypeptide, a small molecule, a peptidomimetic, an siRNA or an antisense oligonucleotide or RNA molecule. In a specific aspect, the miR-25 inhibitor is an miR-25 antagonist.

In another aspect, the subject has heart failure or cardiomyopathy. In a further aspect, the sarcoplasmic reticulum function is improved. In another aspect, damage or failure of contractility of heart muscle is arrested. In another aspect, damage or failure of contractility of cardiomyocytes is arrested. In an additional aspect, administration of the miR-25 inhibitor treats or improves fractional shortening of heart muscle.

In a further aspect, the administration of the miR-25 inhibitor treats or improves heart muscle function as measured by ejection fraction. In an aspect, damage or failure of heart muscle function measured by ejection fraction is arrested. In an additional aspect, administration of the miR-25 inhibitor treats or improves fibrosis of heart muscle. In one aspect, damage or failure of fibrosis of heart muscle is arrested by the administration of the miR-25.

In a further aspect, proteins regulated by miR-25 are modulated following administration of the miR-25 inhibitor. In an additional aspect, the proteins regulated by miR-25 include Acbd4, Adam23, Fbxw7, Lmbr11, Nck2, Plekhm1, Rab8b, SERCA2a, Tmem184b, Ttc39b, Whsc111, Wwp2 and zinc and ring finger 2, or any combination thereof. In another aspect, the levels of SERCA2a are increased after treatment compared to levels prior to administration of the miR-25 inhibitor. In an aspect, the miR-25 inhibitor is administered by oral, transdermal, intravenous, intramuscular, or subcutaneous routes.

In an additional embodiment, the present invention provides method of treating heart failure or cardiomyopathy, comprising administering to a subject a miR-25 inhibitor.

In a further embodiment, the present invention provides a method of increasing SERCA2a levels and function in a subject comprising administration of a miR-25 inhibitor. In another aspect, the subject has heart failure or cardiomyopathy.

In another embodiment, the present invention provides a method of increasing calcium uptake of heart muscle or cardiomyocytes, comprising contacting heart muscle or cardiomyocytes with a miR-25 inhibitor In one aspect, SERCA2a levels are increased following administration of the miR-25 inhibitor.

In an embodiment, the present invention provides, a pharmaceutical composition comprising a miR-25 inhibitor and a pharmaceutically acceptable carrier.

In an additional embodiment, the present invention provides a method of identifying an agent that decreases miR-25 expression or inhibits miR-25 activity comprising measuring expression levels of miR-25 or SERCA2a in a cell; contacting the cell with a test agent; measuring expression levels of miR-25 or SEARCA2a in the cell; and determining if expression levels of miR-25 or SERCA2a have decreased, thereby identifying an agent which decreases miR-25 expression or inhibits miR-25 activity. In one aspect, the cell is a cardiac cell. In a specific aspect, the cardiac cell is from a subject. In a further aspect, the test agent is used for treating a subject.

In a further embodiment, the present invention provides a method of diagnosing a cardiac contractile function disorder in a subject comprising comparing the expression level of miR-25 or SERCA2a in a test sample from the subject to the expression level of miR-25 or SERCA2a in a normal sample; wherein a difference in expression level of miR-25 or SERCA2a is diagnostic of a cardiac contractile disorder.

In an embodiment, the present invention provides a method of monitoring a therapeutic regimen for treating a subject having heart failure by determining a change in expression level of miR-25 or SERCA2a during therapy.

### BRIEF DESCRIPTION OF THE DRAWINGS

FIGS. 1a-1g depict high content screening which identifies miRs that control SERCA2a. (a) Schematic of the target sensor construct; (b) Schematic of the screening workflow, showing microscopy images of positive and negative control wells; (c) Overview of results from screening the whole genome collection of miRs; (d) miR activities plotted as % inhibition relative to siRNA against SERCA2 (x-axis) and p-value from T-test (Y-axis); (e) miRs that inhibited SERCA2-eGFP and slowed the Ca²⁺ transient duration, shown here as CTD₅₀ (decay phase duration time from peak 50% maximal value) in transfected HL-1 cells. Box defines mean±std dev; whiskers=±S^{th} and 95^{th} percentile; dots=outliers. *, **, ***=p-value<0.05, <0.01, <0.001. ns=not significant; (f) frequency distribution of CTD75-25 values and log normal curve and (g) miR-25 expression in human heart failure samples, by Q-PCR. Error bars=s.e.m. **indicates P<0.01 (n=5)

FIGS. 2a-2h shows that miR-25 directly targets SERCA2a and IP3R1 and regulates contractile Ca2+ kinetics. (a and b) miR-25 overexpression on SERCA2 (a) and IP3R1 (b) protein levels; (c-f) Sequences of the putative miR-25(SEQ ID NO: 11) recognition elements in the 3′UTR of SERCA2a (SEQ ID NO: 7) and SERCA2a mutant (SEQ ID NO: 8) (c) and IP3R1 (SEQ ID NO: 9) and IP3R1 mutant (SEQ ID NO: 10) (e) mRNAs and the corresponding alterations made by site-directed mutagenesis and mutation of the putative recognition sites in SERCA2a (d) and IP3R1 (f) reporter constructs. (g) Ca²⁺ transient of HL-1 cells transfected as indicated; (h) High throughput kinetic cytometry analysis of Ca²⁺ transient kinetics during the decay phase (Ca²⁺ transient duration time from peak to 50% maximal value, CTD₅₀) of transfected HL-1 cells. *,# indicate significant difference (p<0.01, one-tailed ANOVA) from scrambled sequence control (*) or miR-25 (#).

FIGS. 3a-3x shows Inhibition of miR-25 normalizes TAC-induced cardiac dysfunction. (a) Experimental protocol for the anti-miR-25 therapy in the mouse TAC model of heart failure; (b) Representative two-dimensional guided M-mode images of the left ventricles from mice at baseline, 2 months post-TAC prior to injection, and from injected mice at 4.5 months post-TAC; (c) Effect of anti-miR-25 injection on echocardiographic indices of left ventricular function: ejection fraction (EF) and fractional shortening (FS) expressed as percentages. The number of animals initiated per cohort is n=4 (sham operated), n=10 (TAC+control anti-miR), and n=4 (anti-miR-25); the numbers analyzed per time point are indicated below reflect deaths in the TAC+control anti-miR group. Statistical differences between anti-miR-25 injected and sham indicated by *; between control and anti-miR-25 indicated by #, and the p-values indicated by the number of symbols, * or #, ** or ##, ***=p-value<0.05, <0.01, <0.001; (d-f) Hemodynamic measurements showing effect of anti-miR-25 injection. Pressure-volume plots of treatment cohorts as indicated (d). Note normalization of hemodynamic indices of end systolic pressure volume relationship (ESPVR, slope of lines in d) (e), and EF (f); (g) Heart weight to body weight ratio showing normalization by anti-miR-25; (h) Q-PCR results showing the effect of treatment on endogenous miR-25 levels; (i) Representative immunoblot images (left) and quantification (right) showing the effect of treatment on endogenous SERCA2a; (j-x) Representative Masson's trichrome (j,k,l,o,p,q,t,u,v) and hematoxylin/eosin (m,n,r,s,w,x) staining results from sham, control and anti-miR-25 treatment groups. Quantification of fibrotic area expressed as proportion (%) of blue area in trichrome stained sections (y) and average cardiomyocyte cross sectional area determined from histological sections (z), see Detailed Methods online.* or #, ** or ##, ***=p-value<0.05, <miR0.01, <0.001. ns=not significant.

FIG. 4 shows miR-25 expression by Q-PCR in mice subjected to aortic constriction (TAC) and 1 month after surgery. * indicates significant difference from unoperated control.

FIGS. 5a-5c shows the effect of siRNA to SERCA2a and miR-25 on Ca²⁺ transient kinetic parameters of HL-1 cells. (a) CTD₅₀ (Ca²⁺ transient duration 50, which is the time from maximal value to 50% maximal value), (b) CTD₇₅₋₂₅ (Ca²⁺ transient duration 75-25, which is the time from 75% maximal value to 25% maximal value) and (c) Vmax upstroke (the maximal velocity upstroke phase of the Ca²⁺ transient). Data are represented as whisker plots, with the box denoting the 25th and 75th percentiles, the whiskers the 5th and 95th percentiles, the middle bar is the median and outliers are indicated as individual dots.

FIGS. 6a-6c shows the effect of siRNA to /P3R1 isoforms and miR-25 on Ca²⁺ transient parameters of HL-1 cells. (a) CTD₅₀ (Ca²⁺ transient duration 50, which is the time from maximal value to 50% maximal value), (b) CTD₇₅₋₂₅ (Ca²⁺ transient duration 75-25, which is the time from 75% maximal value to 25% maximal value) and (c) Vmax upstroke (the maximal velocity upstroke phase of the Ca²⁺ transient). Data are represented as whisker plots, with the box denoting the 25th and 75th percentiles, the whiskers the 5th and 95th percentiles, the middle bar is the median and outliers are indicated as individual dots.

FIG. 7 shows a Kaplan Meier survival curve for anti-miR-25 treatment. Survival probability is plotted over time, showing cumulative protective effect of anti-miR-25 relative to control (scrambled sequence) anti-miR injections following trans aortic constriction (TAC). The summary of two experiments is shown plotting time from injection. Groups were sham-operated (n=8); TAC+anti miR-25 (n=8) and TAC+control (scrambled sequence) anti-miR (n=22).

FIG. 8 shows Echocardiographic (Table 1) and in vivo hemodynamic (Table 2) assessments of cardiac function in mice following TAC.

### DETAILED DESCRIPTION OF THE INVENTION

The present invention is based on the discovery that microRNAs contribute to the loss of cardiac contractility. Specifically, miR-25 is a negative regulator and binds to transcripts encoding SERCA2a which results in a loss of function and interferes with Ca²⁺ handling. Accordingly, the present invention provides methods of increasing cardiac contractile function by inhibiting miR-25. The invention further provides methods to identify agents that can modulate miR-25 activity, including high throughput screening methods, and provides a means to identify agents that are useful for treating patients having cardiac contractile function associated disorders.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, the term “disorder” or “disease” refers to any condition resulting in decreased cardiac contractile function. The term “cardiac contractile function associated disease” or “cardiac contractile function associated disorder” is used herein to refer specifically to a condition in which cardiac contractile function is decreased below the level of cardiac contractile function in a corresponding normal heart cell. Cardiac contractile function associated disorders include, but are not limited to, heart failure, cardiomyopathy, cardiovascular disorders, sleep disorders, obesity, excessive scarring resulting from acute or repetitive traumas, including surgery or radiation therapy, fibrosis of organs including scleroderma, keloids, and hypertrophic scarring.

As used herein, the term “contractile function” refers to the ability of the heart to contract, by which the muscle increases in tension. The normal contractile function of the heart involves a regular contraction and release pattern.

It is well established that the increase in cytosolic Ca²⁺ during systole occurs by release of Ca²⁺ from the sarcoplasmic reticulum through a specialized release channel, the ryanodine receptor, via the process of Ca²⁺-induced Ca²⁺ release (CICR). The entry of a small amount of (“trigger”) Ca²⁺ through the sarcolemmal L-type Ca²⁺ current (I_{Ca}) produces a localized increase of [Ca²⁺]ᵢ in the small space between the surface and SR membranes. This increases the open probability of the RyR2, resulting in the efflux of Ca²⁺ from the SR into the cytoplasm. Amplification of I_{Ca} by CICR elevates myoplasmic Ca²⁺ concentrations to initiate muscle contraction. Relaxation is initiated by a lowering of [Ca²⁺]ᵢ produced either by pumping back Ca²⁺ into the SR by the SR Ca²⁺-ATPase or out or the cell, largely by the sarcolemmal Na⁺—Ca²⁺ exchange.

In many types of heart failure, cardiac myocyte calcium handling is abnormal due to downregulation of key calcium-handling proteins like the Ca²⁺ ATPase of the sarcoplasmic reticulum (SERCA2a) and ryanodine receptor (RyR2). The alteration in SERCA2a and RyR2 expression results in altered cytosolic calcium transients leading to abnormal contraction. An interruption of the heart's ability to release Ca²⁺ from the SR during systole results in decreased contractile function, which is associated with congestive heart failure. Present treatment options for congestive heart failure range from dietary restrictions to cardiac transplantation. The present invention provides an alternate treatment. It has been found that miR-25 levels correlate with decreased cardiac contractility and heart failure in humans and mice. Accordingly, a method of treating or preventing heart failure by administration of a miR-25 inhibitor to a subject to alter contractile function in the subject is provided.

MicroRNAs (miRNA) are single-stranded RNA molecules, which regulate gene expression. miRNAs are encoded by genes from whose DNA they are transcribed but miRNAs are not translated into protein; instead each primary transcript (a pri-miRNA) is processed into a short stem-loop structure called a pre-miRNA and finally into a functional miRNA. Mature miRNA molecules are either fully or partially complementary to one or more messenger RNA (mRNA) molecules, and their main function is to down-regulate gene expression. MicroRNAs can be encoded by independent genes, but also be processed (via the enzyme Dicer) from a variety of different RNA species, including introns, 3′ UTRs of mRNAs, long noncoding RNAs, snoRNAs and transposons. As used herein, microRNAs also include “mimic” microRNAs which are intended to mean a microRNA exogenously introduced into a cell that have the same or substantially the same function as their endogenous counterpart. Thus, while one of skill in the art would understand that an agent may be an exogenously introduced RNA, an agent also includes a compound or the like that increase or decrease expression of microRNA in the cell.

miRs are capable of targeting hundreds of mRNA transcripts, corresponding to hundreds to thousands of proteins, Thus, it is impossible to identify any one miR that night serve as a high level regulator of cardiac contractility, which therefore may be therapeutically useful, without extensive experimentation. Specifically, there are 333 miRs which are predicted to target SERCA2ausing computational methods, e.g. miRNA Data Interpretation Portal (miRDP). By comparing computational information and biochemical information (e.g. Clash, PAR-Clip), it was found that determinations of miR:target interactions using computational approaches had positive and false negative rates of 35%. Thus it was necessary to perform biological screening to discover miRs which potentially block SERCA2a activity and cardiac contractility, particularly in the setting of heart failure.

Accordingly, in one embodiment, the present invention provides a method of increasing contractility of heart muscle or cardiomyocytes in a subject comprising administering a miR-25 inhibitor. In one aspect, the miR-25 inhibitor is selected from the group consisting of an antagonist, a peptide, a polynucleotide, an antibody, a polypeptide, a carbohydrate, a small molecule, a peptidomimetic, a siRNA or an antisense oligonucleotide or RNA molecule. In a specific aspect, the miR-25 inhibitor is an miR-25 antagonist.

In another aspect, the subject has heart failure or cardiomyopathy. In a further aspect, the sarcoplasmic reticulum function is improved. In another aspect, damage or failure of contractility of heart muscle is arrested. In another aspect, damage or failure of contractility of cardiomyocytes is arrested. In an additional aspect, administration of the miR-25 inhibitor treats or improves fractional shortening of heart muscle.

In a further aspect, the administration of the miR-25 inhibitor treats or improves heart muscle function as measured by ejection fraction. In an aspect, damage or failure of heart muscle function measured by ejection fraction is arrested. In an additional aspect, administration of the miR-25 inhibitor treats or improves fibrosis of heart muscle. In one aspect, damage or failure of fibrosis of heart muscle is arrested by the administration of the miR-25.

In a further aspect, proteins regulated by miR-25 are modulated following administration of the miR-25 inhibitor. In an additional aspect, the proteins regulated by miR-25 include Acbd4, Adam23, Fbxw7, Lmbr11, Nck2, Plekhm1, Rab8b, Tmem184b, Ttc39b, Whsc111, Wwp2 and zinc and ring finger 2, or any combination thereof. The modulation of proteins regulated by miR-25 includes increased or decreased expression of the proteins or a combination thereof. In another aspect, the levels of SERCA2a are increased after treatment compared to levels prior to administration of the miR-25 inhibitor. In an aspect, the miR-25 inhibitor is administered by oral, transdermal, intravenous, intramuscular, or subcutaneous routes.

In an additional embodiment, the present invention provides method of treating heart failure or cardiomyopathy, comprising administering to a subject a miR-25 inhibitor.

In a further embodiment, the present invention provides a method of increasing SERCA2a levels and function in a subject comprising administration of a miR-25 inhibitor. In another aspect, the subject has heart failure or cardiomyopathy.

In another embodiment, the present invention provides a method of increasing calcium uptake of heart muscle or cardiomyocytes, comprising contacting heart muscle or cardiomyocytes with a miR-25 inhibitor In one aspect, SERCA2a levels are increased following administration of the miR-25 inhibitor.

The terms “small interfering RNA” and “siRNA” also are used herein to refer to short interfering RNA or silencing RNA, which are a class of short double-stranded RNA molecules that play a variety of biological roles. Most notably, siRNA is involved in the RNA interference (RNAi) pathway where the siRNA interferes with the expression of a specific gene. In addition to their role in the RNAi pathway, siRNAs also act in RNAi-related pathways (e.g., as an antiviral mechanism or in shaping the chromatin structure of a genome).

Polynucleotides of the present invention, such as antisense oligonucleotides and RNA molecules may be of any suitable length. For example, one of skill in the art would understand what lengths are suitable for antisense oligonucleotides or RNA molecule to be used to regulate gene expression. Such molecules are typically from about 5 to 100, 5 to 50, 5 to 45, 5 to 40, 5 to 35, 5 to 30, 5 to 25, 5 to 20, or 10 to 20 nucleotides in length. For example the molecule may be about 5, 10, 15, 16, 17, 18, 19, 20, 21, 22, 23, 24, 25, 26, 27, 28, 29, 30, 31, 32, 33, 34, 35, 40, 45 or 50 nucleotides in length. Such polynucleotides may include from at least about 15 to more than about 120 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In various embodiments antisense oligonucleotides or RNA molecules include oligonucleotides containing modifications. A variety of modification are known in the art and contemplated for use in the present invention. For example oligonucleotides containing modified backbones or non-natural internucleoside linkages are contemplated. As used herein, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides. Examples of polynucleotides and antisense oligonucleotides or RNA molecules which contain modifications are described in detail in U.S. Pat. Nos. 7,683,036; 7,759,319; 8,110,558; 8,178,506; 8,211,867; 8,466,120; and 8,546,350 and U.S. Patent Applicant Nos. 20120148664, 2012184596, 20120238619 and 20130296402, all of which are incorporated by reference in their entirety in the disclosure of this application

Oligonucleotides may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazi-n-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrimido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases are known in the art. Certain of these nucleobases are particularly useful for increasing the binding affinity of the oligomeric compounds described herein. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2 C and are presently preferred base substitutions, even more particularly when combined with 2′-O-methoxyethyl sugar modifications.

In related aspects, the present invention includes use of Locked Nucleic Acids (LNAs) to generate antisense nucleic acids having enhanced affinity and specificity for the target polynucleotide. LNAs are nucleic acid in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2.

Other modifications include 2′-methoxy(2′—O—CH₃), 2′-aminopropoxy(2′-OCH₂CH₂CH₂NH₂), 2′-allyl (2′-CH—CH—CH₂), 2′-O-allyl (2′-O—CH₂—CH—CH₂), 2′-fluoro (2′-F), 2′-amino, 2′-thio, 2′-Omethyl, 2′-methoxymethyl, 2′-propyl, and the like. The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazi-n-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrimido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases are known in the art. Certain of these nucleobases are particularly useful for increasing the binding affinity of the oligomeric compounds described herein. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2 C and are presently preferred base substitutions, even more particularly when combined with 2′-O-methoxyethyl sugar modifications.

Another modification of the antisense oligonucleotides described herein involves chemically linking to the oligonucleotide one or more moieties or conjugates which enhance the activity, cellular distribution or cellular uptake of the oligonucleotide. The antisense oligonucleotides can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., dihexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylaminocarbonyloxycholesterol moiety.

As used herein, “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold; F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttranslational Modifications and Aging”, Ann NY Acad Sci (1992) 663:48-62.

An expression vector (or the polynucleotide) generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb. 25:37-42, 1993; Kirshenbaum et al., J. Clin. Invest. 92:381-387, 1993; each of which is incorporated herein by reference).

Viral expression vectors can be particularly useful for introducing a polynucleotide useful in a method of the invention into a cell, particularly a cell in a subject. Viral vectors provide the advantage that they can infect host cells with relatively high efficiency and can infect specific cell types. For example, a polynucleotide encoding a protein or functional peptide portion thereof can be cloned into a baculovirus vector, which then can be used to infect an insect host cell, thereby providing a means to produce large amounts of the encoded protein or peptide portion. The viral vector also can be derived from a virus that infects cells of an organism of interest, for example, vertebrate host cells such as mammalian, avian or piscine host cells. Viral vectors can be particularly useful for introducing a polynucleotide useful in performing a method of the invention into a target cell. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpes virus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each of which is incorporated herein by reference). In one aspect of the invention, an adenovirus vector is utilized. Adenoviruses are double-stranded DNA viruses, where both strands of DNA encode genes. The genome encodes about thirty proteins. In another aspect of the invention, an adeno-associated virus (AAV) vector is utilized. In certain aspects, the AAV is AAV serotype 6, 7, 8 or 9.

A polynucleotide sequence encoding a protein can be expressed in either prokaryotes or eukaryotes. Hosts can include microbial, yeast, insect and mammalian organisms. Methods of expressing polynucleotides having eukaryotic or viral sequences in prokaryotes are well known in the art, as are biologically functional viral and plasmid DNA vectors capable of expression and replication in a host. Methods for constructing an expression vector containing a polynucleotide of the invention are well known, as are factors to be considered in selecting transcriptional or translational control signals, including, for example, whether the polynucleotide is to be expressed preferentially in a particular cell type or under particular conditions (see, for example, Sambrook et al., supra, 1989).

A variety of host cell/expression vector systems can be utilized to express a polypeptide coding sequence, including, but not limited to, microorganisms such as bacteria transformed with recombinant bacteriophage DNA, plasmid DNA or cosmid DNA expression vectors; yeast cells transformed with recombinant yeast expression vectors; plant cell systems infected with recombinant virus expression vectors such as a cauliflower mosaic virus or tobacco mosaic virus, or transformed with recombinant plasmid expression vector such as a Ti plasmid; insect cells infected with recombinant virus expression vectors such as a baculovirus; animal cell systems infected with recombinant virus expression vectors such as a retrovirus, adenovirus or vaccinia virus vector; and transformed animal cell systems genetically engineered for stable expression. Where the expressed protein is post-translationally modified, for example, by glycosylation, it can be particularly advantageous to select a host cell/expression vector system that can affect the desired modification, for example, a mammalian host cell/expression vector system.

In yeast cells, a number of vectors containing constitutive or inducible promoters can be used (see Ausubel et al., supra, 1987, see chapter 13; Grant et al., Meth. Enzymol. 153:516-544, 1987; Glover, DNA Cloning Vol. II (IRL Press, 1986), see chapter 3; Bitter, Meth. Enzymol. 152:673-684, 1987; see, also, The Molecular Biology of the Yeast Saccharomyces (Eds., Strathern et al., Cold Spring Harbor Laboratory Press, 1982), Vols. I and II). A constitutive yeast promoter such as ADH or LEU2 or an inducible promoter such as GAL can be used (Rothstein, DNA Cloning Vol. II (supra, 1986), chapter 3). Alternatively, vectors can be used which promote integration of foreign DNA sequences into the yeast chromosome.

Eukaryotic systems, particularly mammalian expression systems, allow for proper post-translational modifications of expressed mammalian proteins. Eukaryotic cells which possess the cellular machinery for proper processing of the primary transcript, glycosylation, phosphorylation, and advantageously, plasma membrane insertion of the gene product can be used as host cells for the expression of a protein, or functional peptide portion thereof.

Mammalian cell systems which utilize recombinant viruses or viral elements to direct expression can be engineered. For example, when using adenovirus expression vectors, the polypeptide coding sequence can be ligated to an adenovirus transcription/translation control complex, e.g., the late promoter and tripartite leader sequence; Alternatively, the vaccinia virus 7.5K promoter can be used (Mackett et al., Proc. Natl. Acad. Sci., USA 79:7415-7419, 1982; Mackett et al., J. Virol. 49:857-864, 1984; Panicali et al., Proc. Natl. Acad. Sci., USA 79:4927-4931, 1982). Particularly useful are bovine papilloma virus vectors, which can replicate as extrachromosomal elements (Sarver et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host cell chromosome, thereby yielding a high level of expression. These vectors can be used for stable expression by including a selectable marker in the plasmid, such as, for example, the neo gene. Alternatively, the retroviral genome can be modified for use as a vector capable of introducing and directing the expression of the protein gene in host cells (Cone and Mulligan, Proc. Natl. Acad. Sci., USA 81:6349-6353, 1984). High level expression, can also be achieved using inducible promoters, including, but not limited to, the metallothionein IIA promoter and heat shock promoters.

In an additional embodiment, the present invention provides a method of identifying an agent that decreases miR-25 expression or inhibits miR-25 activity comprising measuring expression levels of miR-25 or SERCA2a in a cell; contacting the cell with a test agent; measuring expression levels of miR-25 or SEARCA2a in the cell; and determining if expression levels of miR-25 or SERCA2a have decreased, thereby identifying an agent which decreases miR-25 expression or inhibits miR-25 activity. In one aspect, the cell is a cardiac cell. In a specific aspect, the cardiac cell is from a subject. In a further aspect, the test agent is used for treating a subject.

In a further embodiment, the present invention provides a method of diagnosing a cardiac contractile function disorder in a subject comprising comparing the expression level of miR-25 or SERCA2a in a test sample from the subject to the expression level of miR-25 or SERCA2a in a normal sample, wherein a difference in expression level of miR-25 or SERCA2a is diagnostic of a cardiac contractile disorder.

In an embodiment, the present invention provides a method of monitoring a therapeutic regimen for treating a subject having heart failure by determining a change in expression level of miR-25 or SERCA2a during therapy.

A screening assay of the invention also provides a means to determine an amount of a particular agent useful for effecting a desired change in miR-25 expression or activity, thereby modulating cardiac contractile function. Such a method can be performed by contacting a sample with different amounts of the same or different test agents or different amounts of the same or different agents previously identified as modulating sorcin expression in the heart of a subject. As such, the methods of the invention can be used to confirm that an agent suspected of having a particular activity, in fact, has the activity, thus providing a means, for example, to standardize the activity of the agent.

A sample that is examined according to a method of the invention can be any sample that contains, or to which can be added, cardiac cells expressing miR-25. In one aspect, the sample is a biological sample, including, for example, a bodily fluid; an extract from a cell, which can be a crude extract or a fractionated extract; a chromosome, an organelle or a cell membrane; a cell; genomic DNA, RNA, or cDNA, which can be in solution or bound to a solid support; a tissue; or a sample of an organ. A biological sample, for example, from a human subject, can be obtained using well known and routine clinical methods (e.g., a biopsy procedure).

As used herein, the term “test agent” means any compound or agent that is being examined for the ability to reduce miR-25 expression or inhibit miR-25 activity. A test agent (and an agent that reduce miR-25 expression or inhibit miR-25 activity identified by a method of the invention) can be any type of molecule, including, for example a peptide, a polynucleotide (including antisense or RNAi), an antibody, a glycoprotein, a carbohydrate, a small organic molecule, or a peptidomimetic.

Where a test agent is identified as reduce miR-25 expression or inhibit miR-25 activity, thereby altering cardiac contractile function, a screening assay of the invention can further include a step of determining an amount by which the agent increases or decreases miR-25 expression or activity. For example, where an agent is identified that reduces miR-25 expression or activity in the heart of a subject, a method of the invention can further include determining an amount by which the agent reduces miR-25 above a basal level in a corresponding normal sample. Such an agent can be identified by measuring the amount of miR-25 in a single sample both before adding the test agent and after adding the test agent, or can be identified for example, using two samples, wherein one sample serves as a control (no test agent added) and the other sample includes the test agent. As such, a method of the invention provides a means to obtain agents or panels of agents that variously reduce miR-25 expression or inhibit miR-25 activity, thereby altering cardiac contractile function.

As used herein, a “corresponding normal sample” is any sample taken from a subject of similar species that is considered healthy or otherwise not suffering from cardiomyopathy or a related disorder. As such, a normal/standard level of miR-25 expression denotes the level of miR-25, SERCA2a, and/or other protein regulated by miR-25 present in a sample from the normal sample. A normal level of miR-25, SERCA2a, and/or other protein regulated by miR-25 can be established by combining body fluids or cell extracts taken from normal healthy subjects, preferably human, with antibody to miR-25 under conditions suitable for miR-25, SERCA2a, and/or other protein regulated by miR-25 expression. Levels of miR-25, SERCA2a, and/other protein regulated by miR-25 in subject, control, and disease samples from biopsied tissues can be compared with the standard values. Deviation between standard and subject values establishes the parameters for diagnosing disease. A normal level of miR-25, SERCA2a, and/or other protein regulated by miR-25 also can be determined as an average value taken from a population of subjects that is considered to be healthy, or is at least free of a cardiac contractile function associated disorder. A variety of protocols including ELISA, RIA, FACS and RNA microarray analysis are useful for measuring levels of miR-25, SERCA2a, and/or other protein regulated by miR-25, and provide a basis for diagnosing altered or abnormal levels of miR-25, SERCA2a, and/or other protein regulated by miR-25

As disclosed herein, the screening methods of the invention provide the advantage that they can be adapted to high throughput analysis and, therefore, can be used to screen combinatorial libraries of test agents in order to identify those agents that can reduce miR-25 expression or inhibit miR-25 activity. Methods for preparing a combinatorial library of molecules that can be tested for a desired activity are well known in the art and include, for example, methods of making a phage display library of peptides, which can be constrained peptides (see, for example, U.S. Pat. Nos. 5,622,699; 5,206,347; Scott and Smith, Science 249:386-390, 1992; Markland et al., Gene 109:13 19, 1991; each of which is incorporated herein by reference); a peptide library (U.S. Pat. No. 5,264,563, which is incorporated herein by reference); a peptidomimetic library (Blondelle et al., Trends Anal. Chem. 14:83 92, 1995; a nucleic acid library (O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference); an oligosaccharide library (York et al., Carb. Res., 285:99 128, 1996; Liang et al., Science, 274:1520 1522, 1996; Ding et al., Adv. Expt. Med. Biol. 376:261 269, 1995; each of which is incorporated herein by reference); a lipoprotein library (de Kruif et al, FEBS Lett. 399:232 236, 1996, which is incorporated herein by reference); a glycoprotein or glycolipid library (Karaoglu et al., J. Cell Biol. 130:567 577, 1995, which is incorporated herein by reference); or a chemical library containing, for example, drugs or other pharmaceutical agents (Gordon et al., J. Med. Chem. 37:1385-1401, 1994; Ecker and Crooke, BioTechnology 13:351-360, 1995; each of which is incorporated herein by reference). Polynucleotides can be particularly useful as agents that can modulate a specific interaction of molecules because nucleic acid molecules having binding specificity for cellular targets, including cellular polypeptides, exist naturally, and because synthetic molecules having such specificity can be readily prepared and identified (see, for example, U.S. Pat. No. 5,750,342, which is incorporated herein by reference).

In performing a screening assay of the invention in a high throughput (or ultra-high throughput) format, isolated cell membranes or intact cells can be used. An advantage of using intact cells is that the method can be used, for example, to identify an agent useful for reducing miR-25 expression or inhibiting miR-25 activity within the cell. Any number of samples (e.g., 96, 1024, 10,000, 100,000, or more) can be examined in parallel using such a method, depending on the particular support used.

A particular advantage to high throughput screening finds application to the design of personalized medicine. For example, a plurality of test agents can be arranged in an array, which can be an addressable array, on a solid support such as a microchip, on a glass slide, on a bead, or in a well, and the cells of a subject (e.g., a biopsy sample) can be contacted with the different test agents to identify one or more agents having desirable characteristics, including, for example, in addition to the ability to for reduce miR-25 expression or inhibit miR-25 activity, minimal or no toxicity to the cell, desirable solubility characteristics, and the like. Consequently, a treatment regimen may be tailored specifically to the individual based upon the subject's levels of miR-25 expression or activity.

An additional advantage of arranging the samples in an array, particularly an addressable array, is that an automated system can be used for adding or removing reagents from one or more of the samples at various times, or for adding different reagents to particular samples. In addition to the convenience of examining multiple test agents and/or samples at the same time, such high throughput assays provide a means for examining duplicate, triplicate, or more aliquots of a single sample, thus increasing the validity of the results obtained, and for examining control samples under the same conditions as the test samples, thus providing an internal standard for comparing results from different assays.

Various protocols may be employed for screening a library of chemical compounds. To some degree, the selection of the appropriate protocol will depend upon the nature of the preparation of the compounds. For example, the compounds may be bound to individual particles, pins, membranes, or the like, where each of the compounds is segregatable. In addition, the amount of compound available will vary, depending upon the method employed for creating the library. Furthermore, depending upon the nature of the attachment of the compound to the support, one may be able to release aliquots of a compound, so as to carry out a series of assays. In addition, the manner in which the compounds are assayed will be affected by the ability to identify the compound which is shown to have activity.

Where the agents are individually located on a surface in a grid, so that at each site of the grid one knows the identification of each agent, one can provide a cellular lawn which is similarly organized as a grid and may be placed in registry with the agents bound to the solid surface. Once the lawn and solid substrate are in registry, one may release the agents from the surface in accordance with the manner in which the agents are attached. After sufficient time for the agents to bind to the proteins on the cellular surface, one may wash the cellular lawn to remove non-specifically bound agents. One or more washings may be involved, where the washings may provide for varying degrees of stringency, depending upon the desired degree of affinity. Since the preparative process can be repeated, a plurality of solid substrates can be prepared, where the same compounds are prepared at the comparable sites, so that the screening could be repeated with the same or different cells to determine the activity of the individual compounds.

In some instances, the identity of the agent can be determined by a nucleic acid tag, using the polymerase chain reaction for amplification of the tag. See, for example, WO93/20242. In this instance, the agents which are active may be determined by taking the lysate and introducing the lysate into a polymerase chain reaction medium comprising primers specific for the nucleic acid tag. Upon expansion, one can sequence the nucleic acid tag or determine its sequence by other means, which will indicate the synthetic procedure used to prepare the agent.

Alternatively, one may have tagged particles where the tags are releasable from the particle and provide a binary code which describes the synthetic procedure for the compounds bound to the particle. See, for example, Ohlmeyer, et al., PNAS USA (1993) 90:10922. These tags can conveniently be a homologous series of alkylene compounds, which can be detected by gas chromatography-electron capture. Depending upon the nature of the linking group, one may provide for partial release from the particles, so that the particles may be used 2 or 3 times before identifying the particular compound.

While for the most part libraries have been discussed, any large group of compounds can be screened analogously, so long as the miR-25 molecule can be joined to each of the compounds. Thus, compounds from different sources, both natural and synthetic, including macrolides, oligopeptides, ribonucleic acids, dendrimers, etc., may also be screened in an analogous manner.

In an embodiment, the present invention provides, a pharmaceutical composition comprising a miR-25 inhibitor and a pharmaceutically acceptable carrier.

miR-25 represents a specific target for the development of anti-heart failure therapeutics. Accordingly, the invention provides methods of using of an agent that can reduce miR-25 expression or inhibit miR-25 activity to treat a cardiac contractile function associated disorder. As such, the methods provide for the administration of a therapeutically effective amount of an agent that reduce miR-25 expression or inhibit miR-25 activity.

For administration to a subject, an agent that reduce miR-25 expression or inhibit miR-25 activity is administered by a route and under conditions that facilitate contact of the agent with the target cell and, if appropriate, entry into the cell. Thus, the agent can be administered to the site of the cells to be treated, or can be administered by any method that provides the target cells with the agent. Furthermore, the agent generally is formulated in a composition (e.g., a pharmaceutical composition) suitable for administration to the subject. As such, the invention provides pharmaceutical compositions containing an agent that reduce miR-25 expression or inhibit miR-25 activity in a pharmaceutically acceptable carrier. As such, the agents are useful as medicaments for treating a subject suffering from heart failure resulting from a cardiac contractile function associated disorder. Further, such a composition -pan include one or more other compounds that, alone or in combination with the agent that reduce miR-25 expression or inhibit miR-25 activity, provides a therapeutic advantage to the subject, for example, an antibiotic if the subject is susceptible to a bacterial infection, one or more additional antiviral agents known to be useful for treating the particular disease or disorder, a nutrient or vitamin or the like, a diagnostic reagent, toxin, a therapeutic agent such as a cancer chemotherapeutic agent, or any other compound as desired, provided the additional compound(s) does not adversely affect the activity of the agent that reduce miR-25 expression or inhibit miR-25 activity or, if the compound does affect the activity of the agent, does so in a manner that is predictable and can be accounted for in formulating the agent.

Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the agent that alters protein-protein interactions that affect hearing and on the route of administration of the composition, which can be, for example, orally or parenterally such as intravenously, and by injection, intubation, or other such method known in the art.

An agent that reduce miR-25 expression or inhibit miR-25 activity can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley et al., Trends Biochem. Sci. 6:77, 1981, each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a composition useful for practicing a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest. 91:2580-2585, 1993, which is incorporated herein by reference). In addition, a polynucleotide agent can be introduced into a cell using, for example, adenovirus-polylysine DNA complexes (see, for example, Michael et al., J. Biol. Chem. 268:6866-6869, 1993, which is incorporated herein by reference).

The route of administration of a pharmaceutical composition containing an agent that reduce miR-25 expression or inhibit miR-25 activity as discussed herein will depend, in part, on the chemical structure of the molecule. Polypeptides and polynucleotides, for example, are not particularly useful when administered orally because they can be degraded in the digestive tract. However, methods for chemically modifying polypeptides, for example, to render them less susceptible to degradation by endogenous proteases or more absorbable through the alimentary tract are well known (see, for example, Blondelle et al., supra, 1995; Ecker and Crook, supra, 1995). In addition, a peptide agent can be prepared using D-amino acids, or can contain one or more domains based on peptidomimetics, which are organic molecules that mimic the structure of peptide domain; or based on a peptoid such as a vinylogous peptoid.

The pharmaceutical composition as disclosed herein can be administered to an individual by various routes including, for example, orally or parenterally, such as intravenously, intramuscularly, subcutaneously, intraorbitally, intracapsularly, intraperitoneally, intrarectally, intracisternally or by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. A pharmaceutical composition also can be administered to the site of a pathologic condition, for example, intravenously orintra-arterially into a blood vessel supplying a tissue or organ comprising retrovirus infected cells

The pharmaceutical composition also can be formulated for oral formulation, such as a tablet, or a solution or suspension form; or can comprise an admixture with an organic ox inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

The present invention also provides methods for diagnosing cardiac contractile function associated disorders in a subject. In one embodiment, agents identified as reduce miR-25 expression or inhibit miR-25 activity may be used for the diagnosis of conditions or diseases characterized by cardiac contractile function associated disorders, or in assays to monitor patients being treated for heart failure. The agents useful for diagnostic purposes may be prepared in the same manner as those described above for therapeutics. Diagnostic assays for cardiac contractile function associated disorders include methods which utilize the identified agents and a label to detect miR-25 expression m samples such as human body fluids or extracts of cells or tissues. The agents may be used with or without modification, and may be labeled by joining them, either covalently or non-covalently, with a reporter molecule. A wide variety of reporter molecules which are known in the art may be used.

The total amount of an agent that reduce miR-25 expression or inhibit miR-25 activity to be administered in practicing a method of the invention can be administered to a subject as a single dose, either as a bolus or by infusion over a relatively short period of time, or can be administered using a fractionated treatment protocol, in which multiple doses are administered over a prolonged period of time.

Once disease is established and a treatment protocol is initiated, screening assays of the invention may be repeated on a regular basis to evaluate whether the level of miR-25 expression is reduced or miR-25 activity is inhibited and/or cardiac contractile function in the patient begins to approximate that which is observed in the normal patient. The results obtained from successive assays may be used to show the efficacy of treatment over a period ranging from several days to months. Accordingly, the invention is also directed to methods for monitoring a therapeutic regimen for treating a subject having heart failure. A comparison of the cardiac contractile function prior to and during therapy indicates the efficacy of the therapy. Therefore, one skilled in the art will be able to recognize and adjust the therapeutic approach as needed

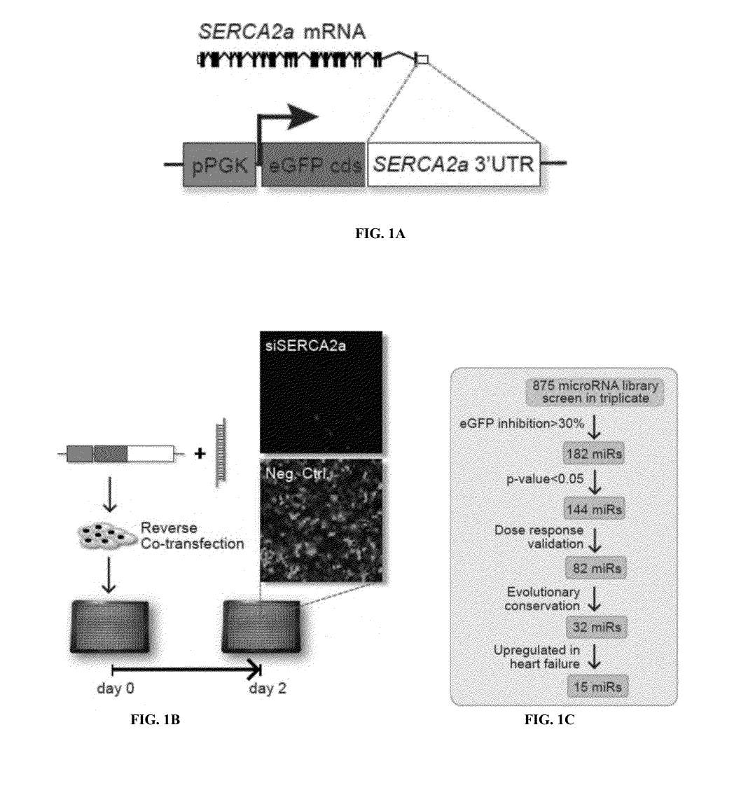

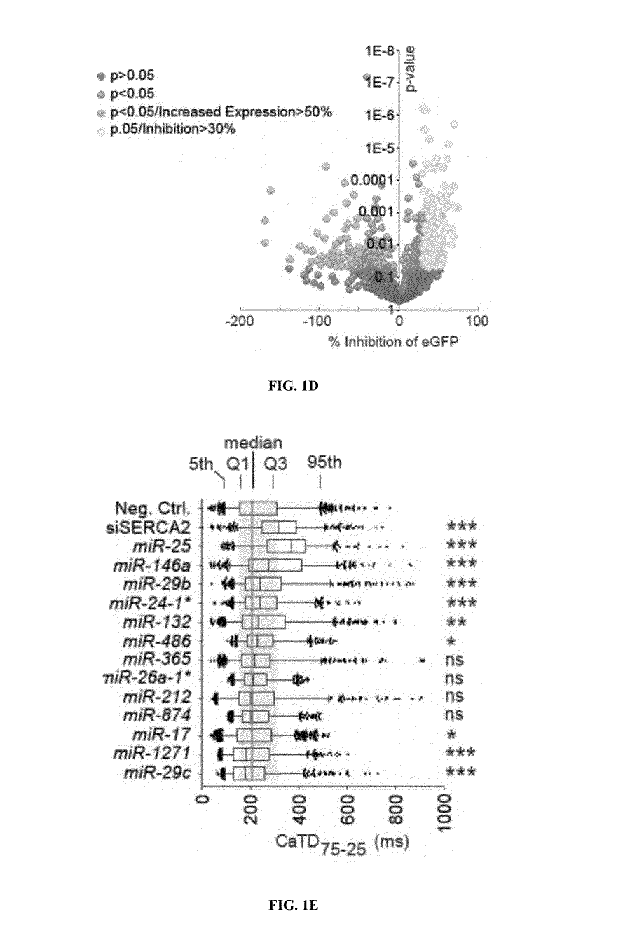

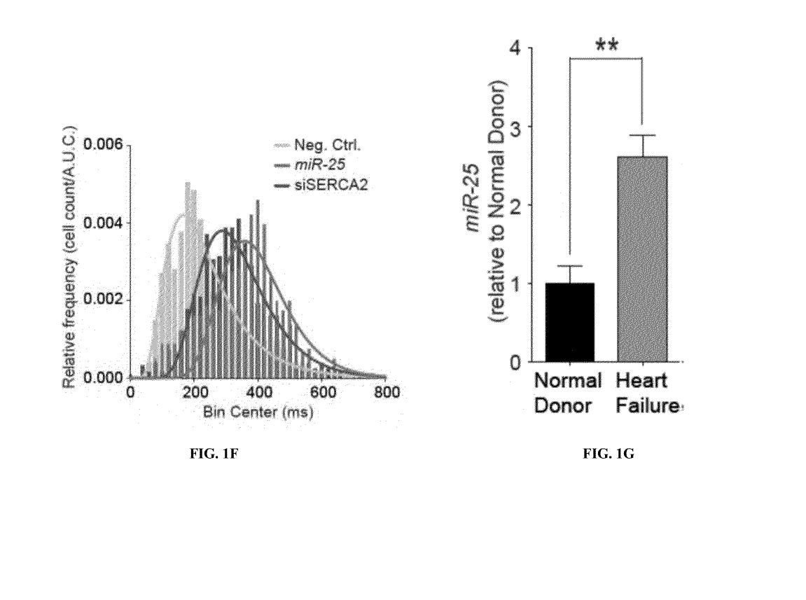

These studies identified miR-25 as a critical regulator of cardiac function during heart failure. miR-25 is upregulated in the failing heart, both in human samples (FIG. 1f) and in the murine TAC-induced model (FIG. 5). Intravenous injection of anti-miR-25 depleted detectable endogenous miR-25 and normalized the levels of SERCA2a in the murine TAC model. Although additional post-transcriptional mechanisms contribute to the decline in SERCA2a function in the failing heart, such as a decrease in SUMOylation that decreases transporter stability and ATPase activity, our observation that anti-miR-25 is sufficient to normalize SERCA2a levels suggests that endogenous miR-25 plays an essential role in controlling the amount of SERCA2a in the failing heart. Since decreased expression and reduced activity of SERCA2a is a major contributor to the decline in cardiac function during heart failure, and normalization of SERCA2a activity by gene delivery elicits comparable effects as was observed with anti-miR-25 injection, the experiments herein provide compelling evidence that miR-25 control of SERCA2a is critical component of the mechanism that downregulates cardiac function in the failing heart. It is well known that microRNAs target multiple proteins, and often proteins that function coordinately in a biological process (Bartel, D. P. MicroRNAs: target recognition and regulatory functions. Cell 136, 215-233, doi:10.1016/j.cell.2009.01.002 (2009) and Filipowicz, W., Bhattacharyya, S. N. & Sonenberg, N. Mechanisms of post-transcriptional regulation by microRNAs: are the answers in sight? Nature reviews. Genetics 9, 102-114, doi:10.1038/nrg2290 (2008)). Thus, blocking miR-25 could exert a therapeutic effect through regulation of other proteins involved in calcium handling within cardiomyocytes, Indeed, Another potential mediator is NAD(P)H oxidase-4 (Nox4) shown recently to be downregulated by miR-25 in a mouse model of diabetic nephropathy. It was found that anti-miR-25 injection increased endogenous Nox4 protein levels in vivo (not shown). Whether Nox4, which is responsible for the production of superoxide, contributes to the salutary effects of miR-25 is unclear since it is reported to confer both protective and detrimental effects that probably depending on the levels and particular reactive oxygen species generated.

In summary, in this study large-scale functional screening of miRs was used and it was found that miR-25 directly controls the production of SERCA2a. Moreover, direct antagonism of miR-25 restores SERCA2a levels and the overall contractile properties of the heart. In addition, the long-term beneficial effects in the mouse model suggest that inhibition of miR-25 may be a novel therapeutic strategy for the treatment of heart failure.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### miR Screening

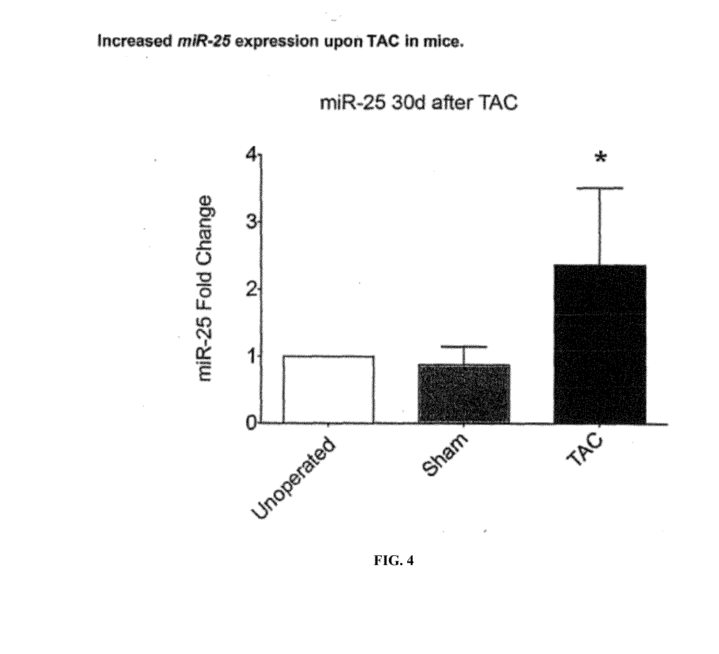

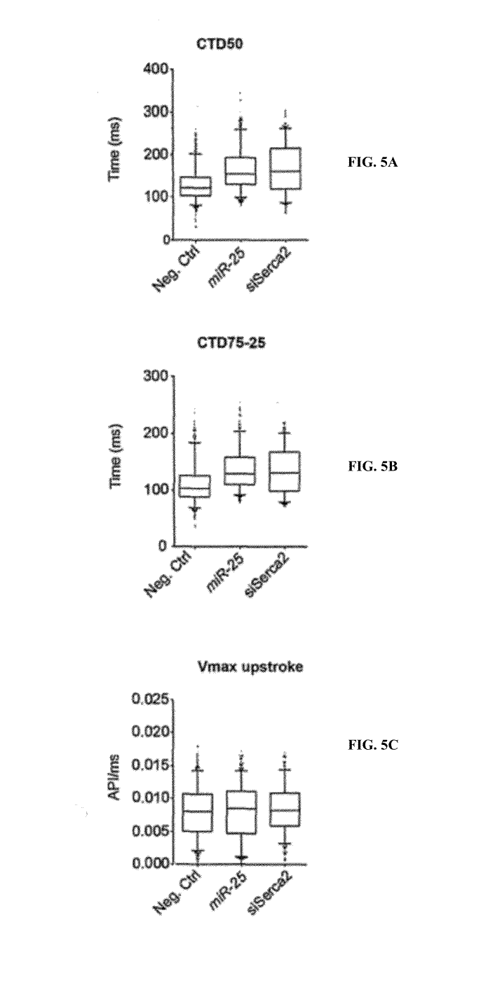

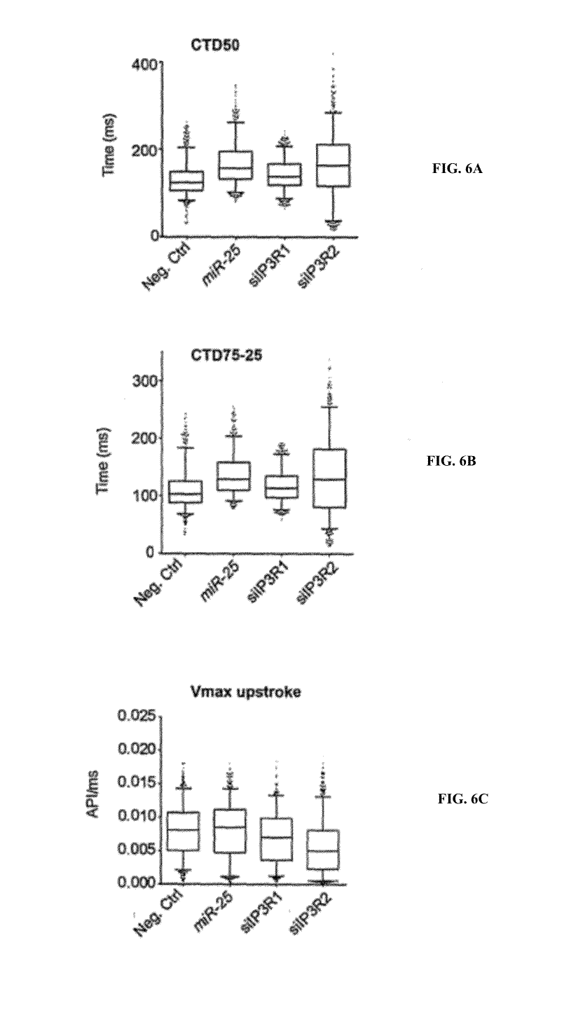

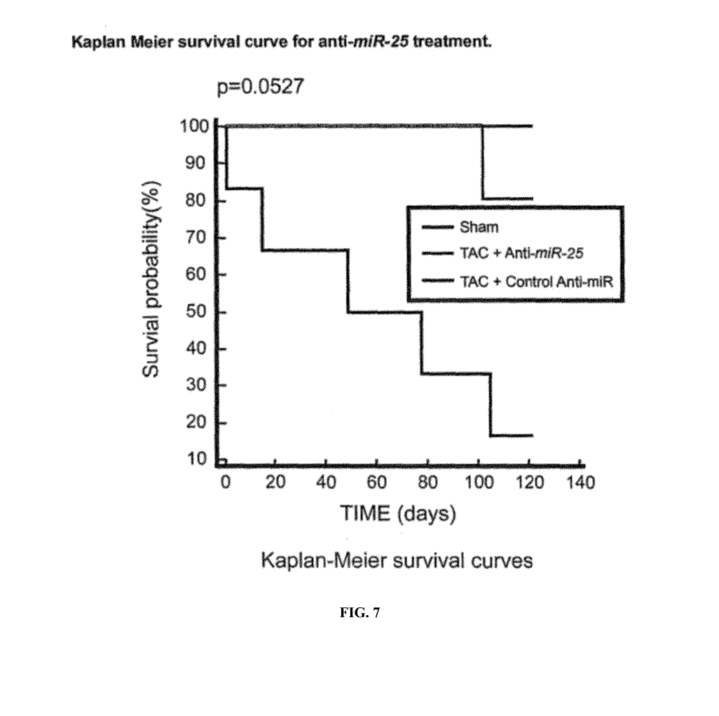

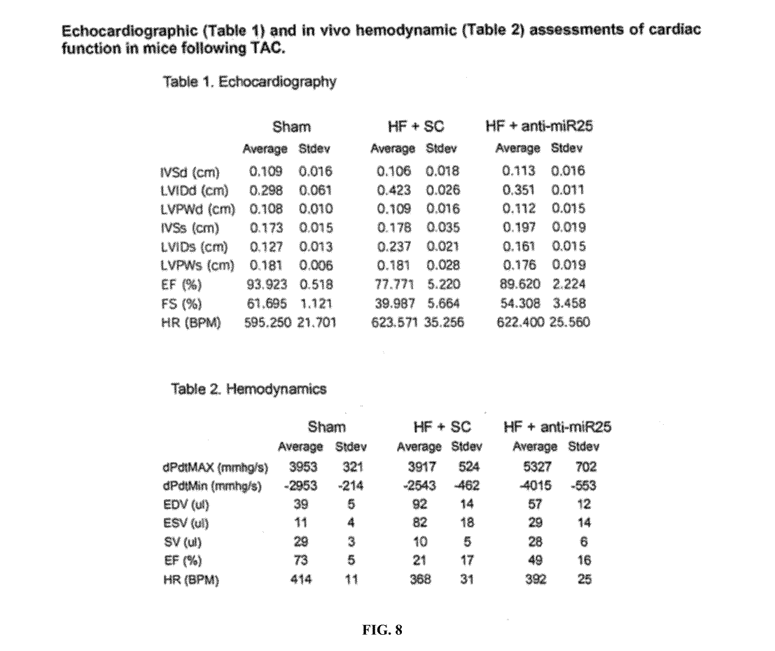

The calcium-transporting ATPase SERCA2a, also known as ATP2A2, is the primary mechanism for Ca²⁺ uptake during excitation-contraction coupling in cardiomyocytes. Impaired Ca²⁺ uptake resulting from decreased expression and reduced activity of SERCA2a is a hallmark of heart failure. Accordingly, restoration of SERCA2a by gene transfer has proven effective in improving key parameters of heart failure in animal models and more recently in clinical trials. Reasoning that certain miRs that downregulate SERCA2 might be elevated in heart failure and compromise cardiac function, an assay was developed to functionally screen a whole-genome collection of miRs for selective downregulation of the Ca²⁺ pump. To perform the functional screen, the SERCA2a mRNA 3′UTR and coding regions were fused downstream of an eGFP coding region, making a “target sensor” construct to permit detection of active miRs by a decrease of eGFP fluorescence (FIG. 1a). 144 miRs reduced eGFP fluorescence by >30% with p<0.05. 49 were evolutionarily conserved and 10 were both evolutionarily conserved and reported to be upregulated in human heart failure (FIG. 1b,c). Of these, 6 cause a highly significant change in the decay phase of the Ca²⁺ transient, shown here as duration from peak to 50% max value, CTD₅₀ (FIG. 1d), as measured in the cardiomyocyte HL-1 cell line using an automated platform capable of recording physiological parameters simultaneously from hundreds of cardiomyocytes in multiwell dishes. The most potent was miR-25, which was confirmed to be upregulated in ventricular myocardium following thoracic aortic constriction in mice (FIG. 4) and in human myocardial samples from patients with severe heart failure at the time of cardiac transplantation (FIG. 1e).

### Example 2

### Effect of miR-25 on Cardiac Contractility

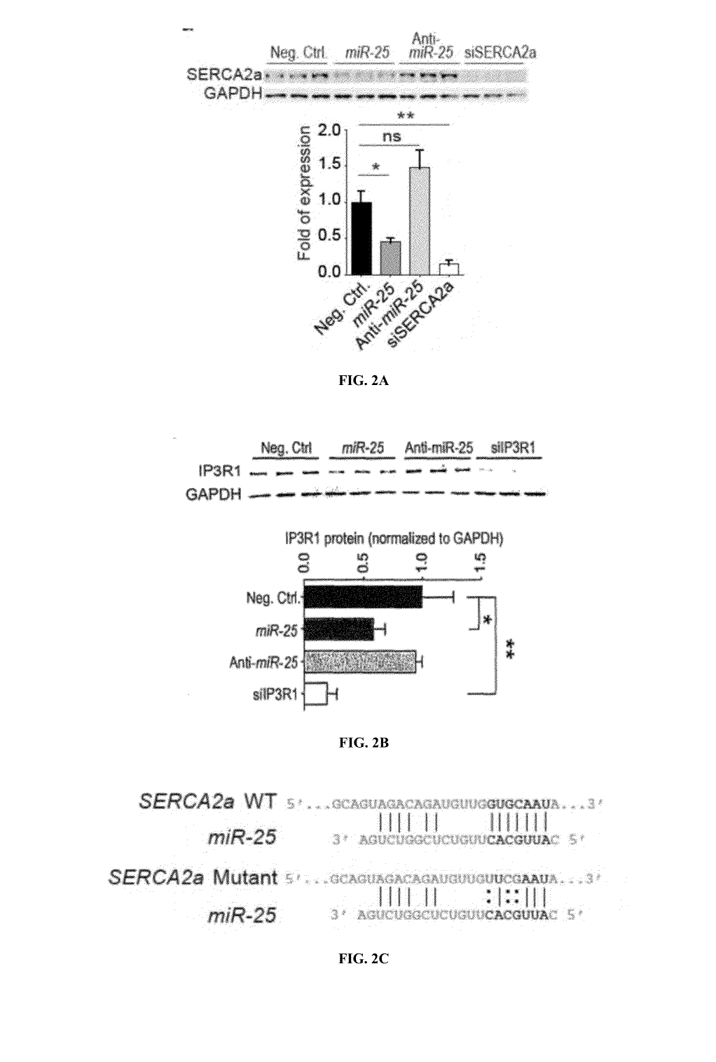

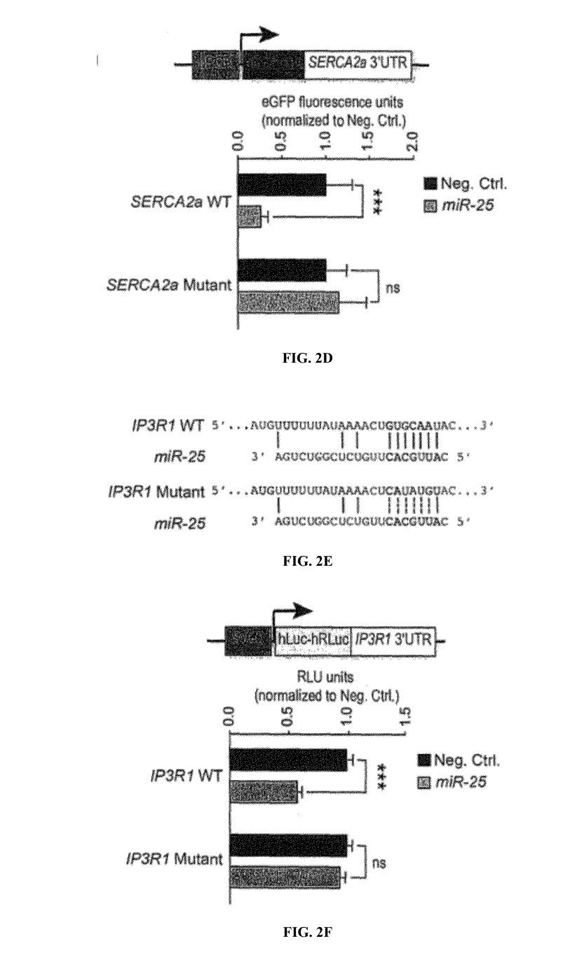

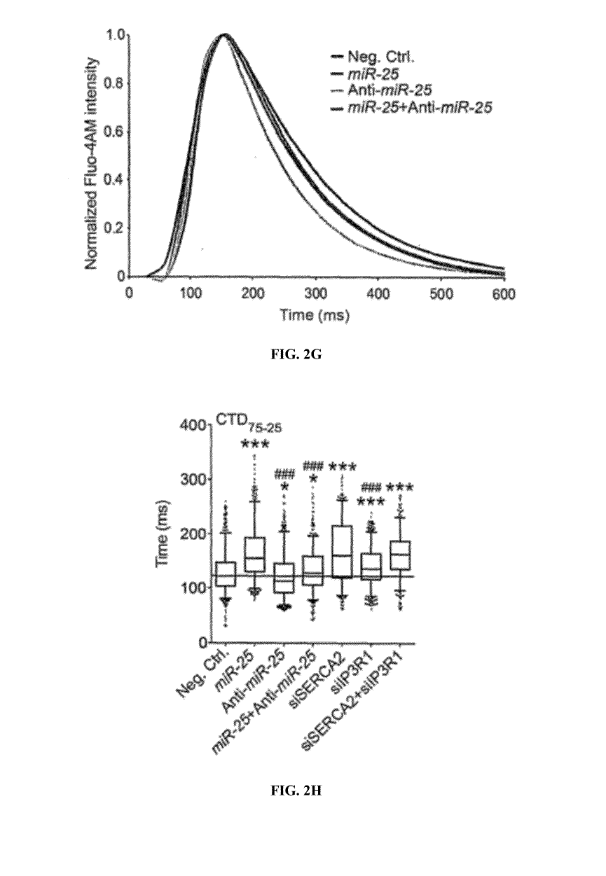

To establish a link between miR-25 and cardiac function, putative protein targets involved in Ca²⁺ handling targets were identified by computational algorithms. In addition to SERCA2a, these included calmodulin, Cav1.2, calmodulin, and the inositol-3′-phosphate receptor-1 (IP3R1). Evaluating these proteins, it was found that overexpression by transient transfection in HL-1 cells selectively downregulated SERCA2 and IP3R1 (FIG. 2a,b), but not the other candidates. miRs bind cognate mRNAs by Watson-Crick base pairing of their “seed” sequence to the specific recognition elements. Based on seed sequence homology, single putative miR-25 recognition sites were identified in SERCA2a and IP3R1 (FIG. 2c,e). To determine if these sites are indeed responsible for miR-25 interactions, the sequences were altered by site-directed mutagenesis and the resulting SERCA2 and IP3R1 constructs evaluated for downregulation by miR-25. Mutation abolished the ability of miR-25 to inhibit reporter expression (FIG. 2d,f), further supporting selective miR-25 interactions with the mRNAs, and indicating that the single recognition elements identified in the 3′UTRs of the SERCA2a and IP3R1 are sufficient for miR-25 activity.

### Example 3

### Inhibition of miR-25 Activity

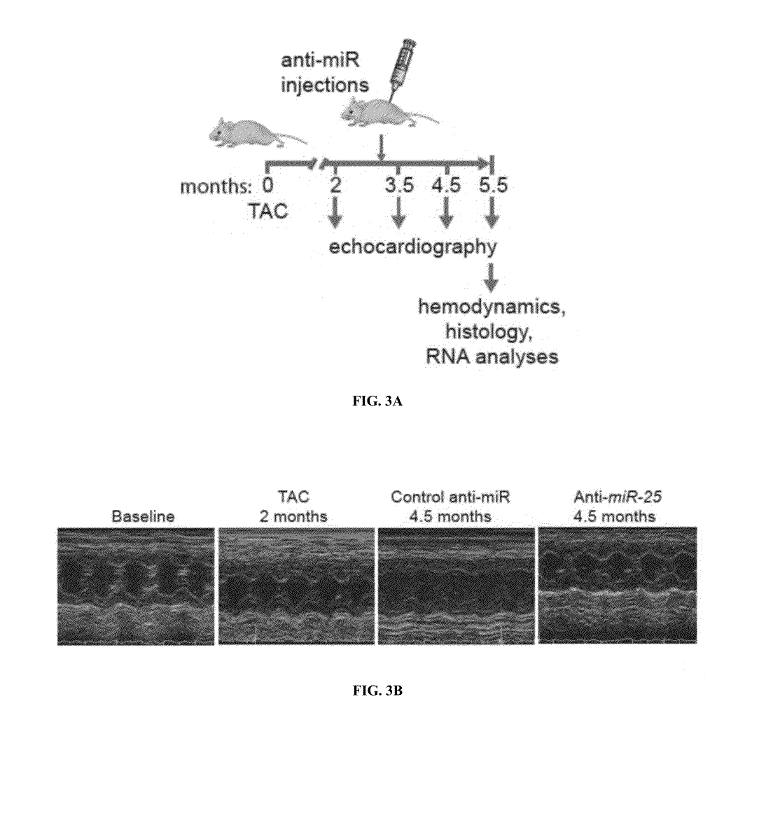

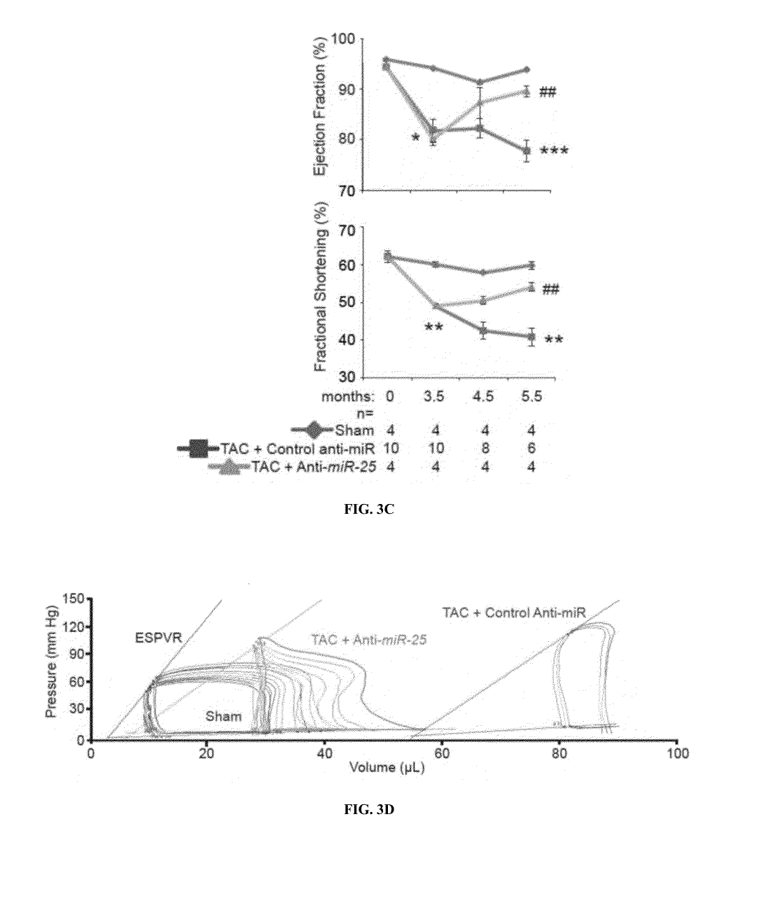

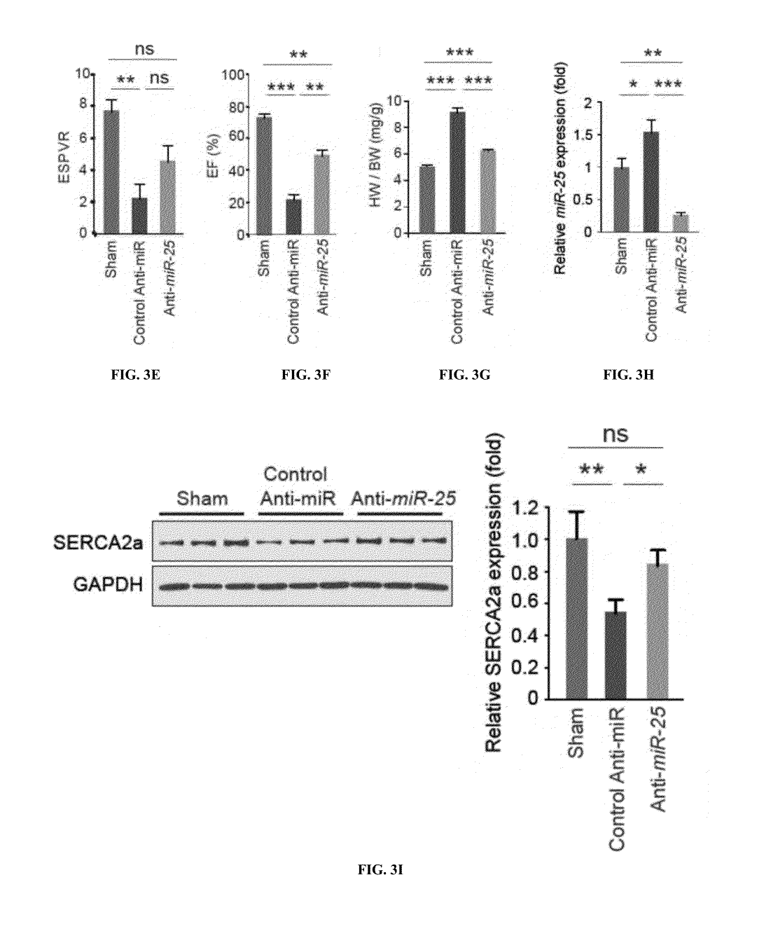

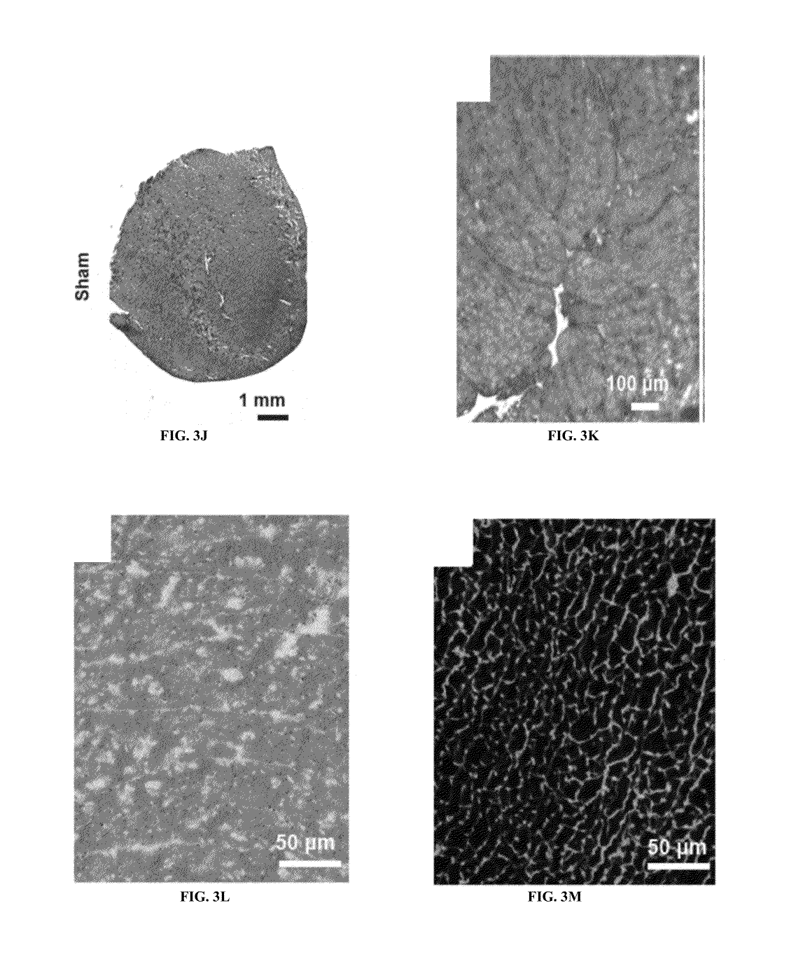

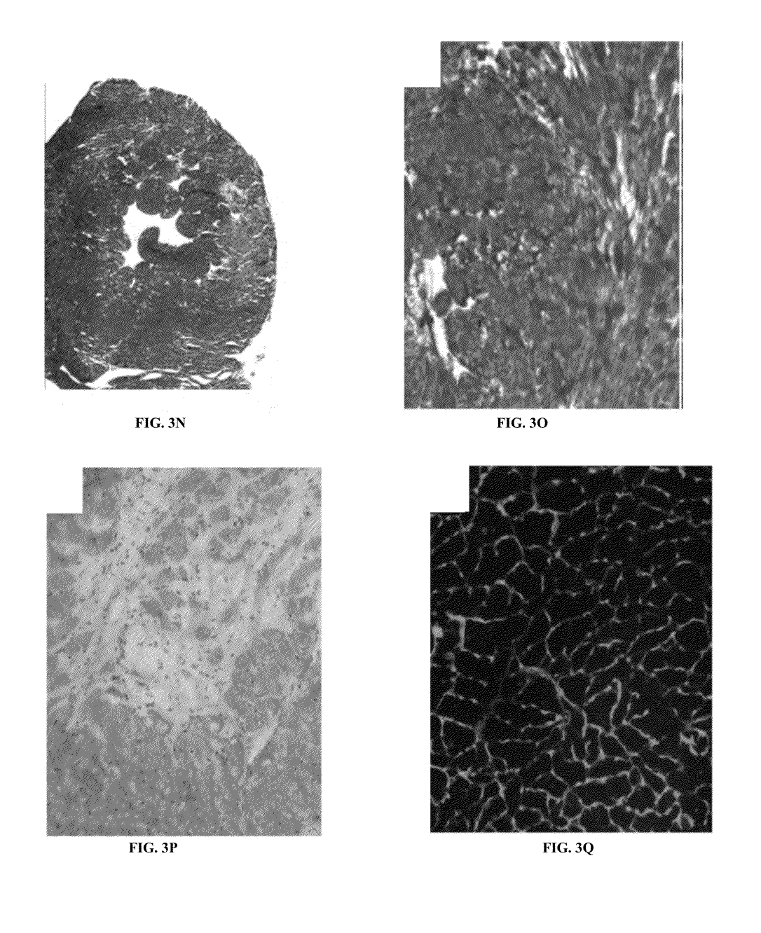

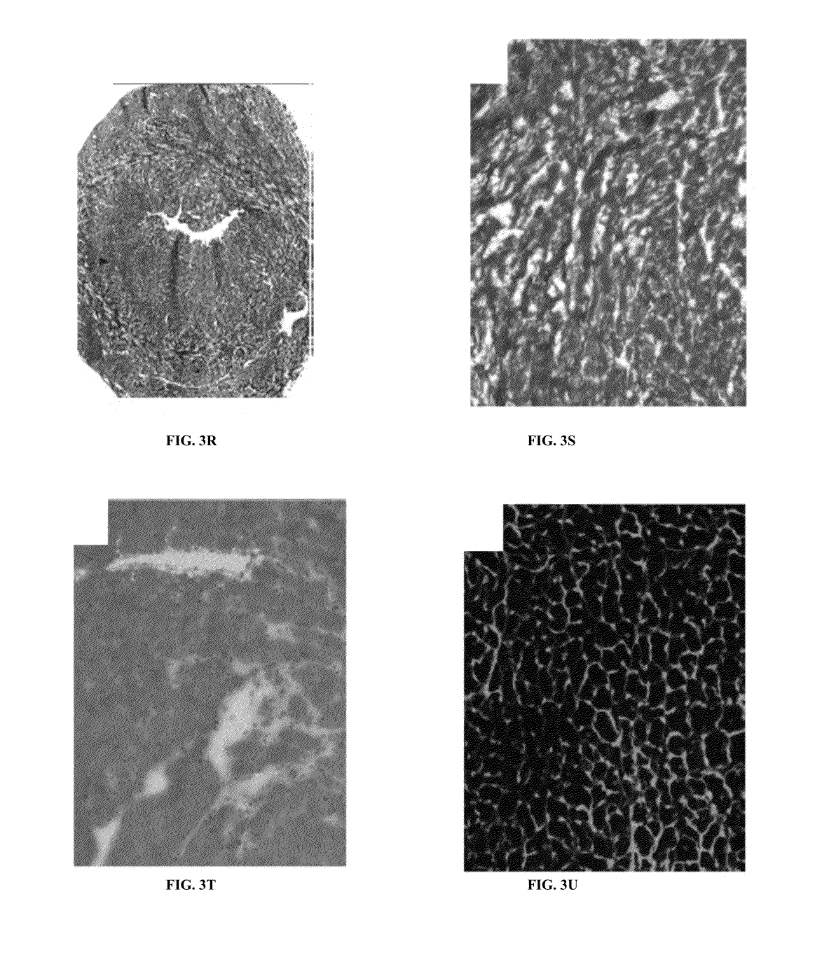

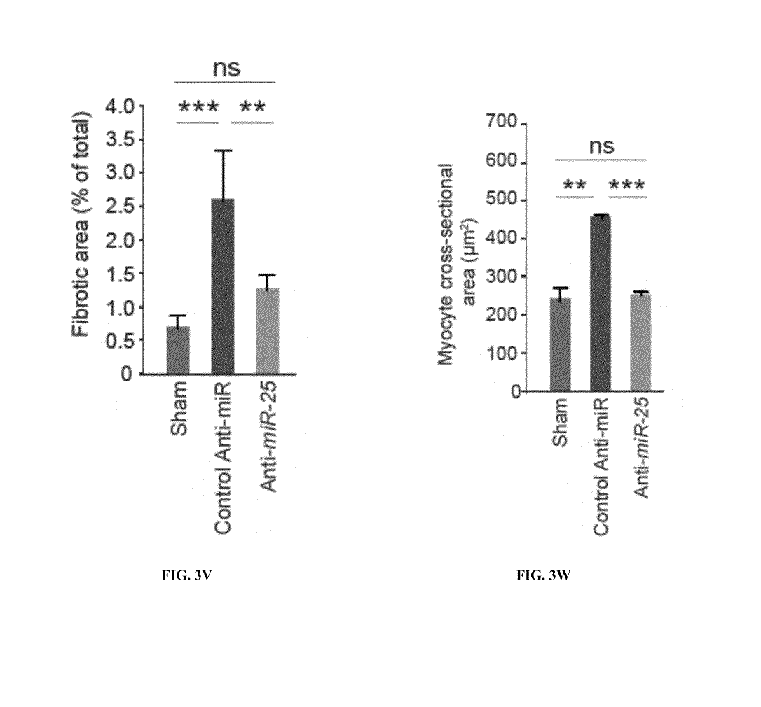

Having shown that SERCA2a and IP3R1 are regulated by miR-25, it was next evaluated whether siRNAs directed specifically against these proteins could mimic the effect of miR-25 on cardiac Ca²⁺ transient kinetics using HL-1 cells. Transient transfection of siRNA against SERCA2a significantly slowed the uptake phase of the Ca²⁺ transient (CTD₅₀) relative to control (scrambled sequence) siRNA (FIG. 2g,h). siRNA against IP3R1 did not significantly affect Ca²⁺ transient kinetics (CTD₅₀, full width half maximum, nor Ca²⁺ transient upstroke Vₘₐₓ, (FIG. 3). Combining both siRNAs was not significantly different than siRNA to SERCA2a alone, suggesting that the predominant effect on Ca²⁺ transient kinetics is mediated through downregulation of SERCA2a.

The physiological effect of antagonizing miR-25 was evaluated. AntagomiRs are antisense oligonucleotides modified to enhance duplex stability and have been used effectively to abrogate the effect of miRs in vitro and in vivo. Anti-miR-25 transfection alone decreased the Ca²⁺ transient duration (decreased CTD₅₀) (FIG. 2g,h). Whereas miR-25 increased CTD₅₀, co-transfection with anti-miR-25 completely cancelled this physiological effect (FIG. 2g,h).

To characterize the potential physiological effects of miR-25 during heart failure, mice were subjected to 3 months of thoracic aortic constriction (TAC) to chronically increase LV load and cause LV dilation. At this point, anti-miR-25 or control (scrambled sequence) anti-miR formulated with in vivo-jetPEI™ reagent mixture were intravenously injected for 3 consecutive days followed by an additional anti-miR-25 injections every week for next 3 weeks, and subsequently monitored for effects on heart function (FIG. 3a and Methods). Functional assessment by echocardiography revealed substantial improvements following injection of anti-miR-25 at 4.5 and 5.5 months after TAC, despite constant pressure overload, compared with severe deterioration in animals injected with the scrambled sequence anti-miR control (FIG. 3b,c and Table 1). Furthermore, hemodynamic analyses at the termination of the studies (5.5. months post-TAC) showed substantially improved LV function in the anti-miR-25-injected mice, effectively restoring the load-independent parameter end systolic pressure volume relationship (ESPVR) and EF to normal levels (FIG. 3d-f). The heart-weight to body-weight ratio was also stabilized (FIG. 3g). Survival was improved, with 8/8 anti-miR-25-injected and 8/8 sham-operated animals surviving compared to 7/14 of the scrambled sequence control anti-miR-injected animals (overall summation of two experiments). Consistent with improved cardiac function, endogenous miR-25 levels were significantly reduced in the anti-miR-25-injected mice relative to controls (FIG. 3h), and SERCA2a levels were significantly increased, indicating that anti-miR-25-injection maintained SERCA2a protein levels despite heart failure (FIG. 3i). Furthermore, injection of anti-miR-25 reduced fibrosis, visible histologically (FIG. 3j-x) and quantified as the proportion (%) fibrotic (blue) area in the Masson's trichrome stained sections for each treatment group (sham, scrambled sequence control anti-miR, anti-miR-25) (FIG. 3y). Finally, anti-miR-25-injection also normalized cardiomyocyte cell size (FIG. 3z). These data provide the first evidence that cardiac transfection of an anti-miR that directly controls cardiac SERCA2a protein levels and engenders long-term improvement of cardiac function.

### Example 4

### Methods

Primary Screening.

For microRNA screening, HEK293 cells were co-transfected with the Ambion® Pre-miR™ miRNA Precursor Human V2.0 microRNA library and 300 ng Serca2a 3′UTR target sensor plasmid (FIG. 1a) per well in 384-well plates (Greiner), in triplicate, using Lipofectamine 2000 (Invitrogen) following manufacturer's protocol. 48 hours after transfection, cells were fixed in 4% paraformaldehyde and imaged (InCell Analyzer 1000; GE Healthcare) and analyzed using CyteSeer software (Vala Sciences) by quantifying Total Integrated Pixel Intensity within an eGFP-positive area.

Kinetic Imaging Cytometry.

HL-1 cells were seeded at a density of 25,000 cells/well on 96-well glass bottom plates (Greiner) and transfected as above. 72 hours post-transfection, cells were loaded with 200 ng/ml Hoechst 33342 (Sigma) and Fluo-4 for 30 minutes at 37^{C} followed by 30 minutes at room temperature (see detailed Methods in online Supplementary Information). Ca²⁺ transient recordings (10 seconds, 33 frames per second) were acquired using a Kinetic Image Cytometer IC 100 (Vala Sciences). Cytometric calcium kinetic parameters were determined by image analysis using CyteSeer software (Vala Sciences) and unresponsive or low responding cells were removed by gating.

Animals.

All mice were housed and treated in accordance with guidelines from the NIH and institutional animal care and use committees, and the protocols used were approved by the Mount Sinai School of Medicine animal care and use committee.

Human Heart Samples.

Left ventricular samples were obtained from severe heart failure samples obtained at the time of cardiac transplantation. Non-failing hearts (which were used as controls) were obtained who died of cerebrovascular accidents with no evidence of contractile dysfunction by echocardiography. The 5 non-failing hearts (3 males and 2 females) had a median age of 43. The 5 heart-failure patients (4 males and 1 female) had a median age of 54 and their mean ejection fraction prior to cardiac transplantation was 22±3%.

Cell Culture.

HEK293 cells were maintained in DMEM/F12 supplemented with 10% FBS, 100 units/mi penicillin and 100 ug/ml streptomycin. HL-1 cells were maintained in Claycomb Medium (Sigma) supplemented with 10% FBS (Sigma), 100 units/ml penicillin, 100 ug/ml streptomycin, 2 mM L-glutamine, 0.1 mM norepinephrine (Sigma) and passaged approximately every 3-4 days when cells reached confluency and spontaneous contractions were observed. For transient transfection of both cell lines, Lipofectamine 2000 (Invitrogen) was used following manufacturer's protocol. Cy3 labeled siRNA or miR was used for control in all transfection experiments.

Target Sensor.

To screen for microRNA repression of the cardiac specific Serca2a isoform the CMV promoter of the pDsRed-N1 vector (Clontech) was substituted with the hPGK promoter driving expression of eGFP. DsRed sequence was replaced with the human Serca2a 3′ UTR sequence, which was obtained by PCR of differentiated human ES cells displaying spontaneous contractions. Primer sequences used for Serca2a amplification were as follows:

hSerca2a F:
(SEQ ID NO: 1)
5′-CGGGGTACCTGCAATACTGGAGTAACCGCTTC-3′
hSerca2a R:
(SEQ ID NO: 2)
5′-CGGCGGCCGCATTTACCTGAAACCATGTCTGTGC-3′

microRNA Screen.

HEK293 cells were co-transfected with the Ambion® Pre-miR™ miRNA Precursor Human V2.0 microRNA library and 300 ng Serca2a 3′UTR target sensor plasmid per well in 384-well plates (Greiner). Transfections were performed in triplicate. At 48 h after transfection cells were fixed in 4% paraformaldehyde and imaging was performed using an automated fluorescent microscope (InCell Analyzer 1000; GE Healthcare) and analyzed using CyteSeer software (Vala Sciences) by quantifying Total Integrated Pixel Intensity within an eGFP-positive area, as described (Colas et al., Genes Dev. 26:2567 (2012); McKeithan et al., Current Protocols in Stem Cell Biol. Chapter 1:Unit 1F (2012)).

Site-Directed Mutagenesis.

Site-directed mutagenesis was used to modify the miR-25 seed binding sequence using Pfu Turbo DNA Polymerase. Dpn I was used to digest non-mutated DNA template before transforming the mutated plasmids. Primers used were as follows:

Serca2a 3′UTR F:
(SEQ ID NO: 3)
5′-GCAGTAGACAGATGTTGTTCGAATACAAATATTGTGATGC-3′
Serca2a 3′UTR R:
(SEQ ID NO: 4)
5′-GCATCACAATATTTGTATTCGAACAACATGTGTCTACTGC-3′
IP3R1 3′UTR F:
(SEQ ID NO: 5)
5′-ATGTTTTTTATAAAACTCATATGTACGAATTATGCAATCAC-3′
IP3R1 3′UTR R:
(SEQ ID NO: 6)
5′-GTGATTGCATAATTCGTACATATGAGTTTTATAAAAAACAT-3′

The mutated sequences were designed to contain restriction enzyme recognition sites (marked in bold) used to verify correct mutation of the seed site.

IP3R1 Target Sensor Luciferase Assay.

HEK293 cells were co-transfected with microRNA and 100 ng human IP3R1 Type 1 miTarget (GeneCopoeia). 48 h after transfection reporter activity was analyzed using the Dual-Glo® Luciferase Assay System (Promega) and EnVision plate reader (PerkinElmer). Data are presented as a ratio of firefly luciferase activity normalized to Renilla luciferase.

Quantitative RT-PCR.

RNA was isolated from human tissue using Trizol (Invitrogen) according to manufacturer's protocol. All-in-One™ miRNA qRT-PCR Detection Kit (GeneCopoeia) was used for both cDNA synthesis and quantitative detection using miR-25 specific primers (GeneCopoeia). Rnu6 was used for normalization.

Protein Expression Analyses.

For in vitro transfection experiments using HL-1 cells, samples were lysed 72 h after transfection using Novex Tris-Glycine 2× Sample Buffer supplemented with 5% β-mercaptoethanol at 55^{C} for 1 h. Samples were loaded on SDS-polyacrylamide electrophoresis gels for separation, followed by blotting of protein onto polyvinylidene fluoride membrane. Membranes were then incubated as follows: Serca2a (1:2000; 21st Century Biochemicals), IP3R1 (1:200, goat polyclonal, sc-6093; Santa Cruz Biotechnology), Calmodulin (1:2000, rabbit monoclonal, ab45689; Abcam), Cav1.2 (1:100, rabbit polyclonal, sd-16229-R; Santa Cruz Biotechnology), GAPDH (1:1000), and NOX4 (1:500). Alexafluor-labeled secondary antibodies were used (1:10,000) for detection of the specific bands. Quantitative analysis was done using the Odyssey® imaging system (LI-COR Biosciences).

Calcium Imaging and Analysis.

HL-1 cells were seeded at a density of 25,000 cells/well on 96-well glass bottom plates (Greiner) and transfected as described for Cell Culture above. 72 h post-transfection, cells were loaded with 200 ng/ml Hoechst 33342 (Sigma) and Fluo-4 for 30 minutes at 37^{C} followed by 30 minutes at room temperature. Fluo-4 NW Calcium Assay Kit (Invitrogen) was prepared according to manufacturers' instructions in 1× Hanks Balanced Salt Solution containing 20 mM HEPES buffer (Invitrogen) and 2.5 mM Probenecid (Invitrogen). Following loading, cells were incubated with Tyrode's solution (140 mM NaCl, 6 mM KCl, 1 mM MgCl2, 5 mM HEPES, 2 mM CaCl2, 10 mM glucose, pH 7.4) for imaging. Ca²⁺ transient recordings were done using a Kinetic Image Cytometer IC 100 (Vala Sciences). Video streams of 10 seconds of the Fluo-4 green channel were collected for each well at 33 frames per second. Autofocus by the IC 100 was performed automatically by video acquisition using the nuclear channel. All image capture was performed with a 20×0.50 numerical aperture (NA) objective. Cytometric calcium kinetic parameters were determined by image analysis using CyteSeer software (Vala Sciences) and unresponsive or low responding cells were removed by gating.

Bioinformatics.

Predicted miR-25 target binding sites were obtained from TargetScan Human v6.2 (www.targetscan.org). Potential targets of miR-25 involved in calcium signaling pathways in were identified using DianaLab miRPath with TargetScan Mouse v5.0 prediction software (www.diana.cslab.ece.ntua.gr/pathways).

Statistical analysis. Data are presented as mean±standard deviation (SD). Whisker box plots show outliers beyond the 5^{th} and 95^{th} percentiles. Unpaired Student's t-test was performed using GraphPad prism software.

TAC (Trans aortic constriction) surgery. Male mice(B6C3F1 strain) of 8 to 10 weeks of age (25-30 g) were used. The animals were anesthetized with Ketamine (95 mg/kg)/xylazine (5 mg/kg) administered via intraperitoneal injection. The mice were ventilated with a tidal volume of 0.1 ml and a respiratory rate of 110 breaths per minute (Harvard Apparatus). A longitudinal incision of 2 to 3 mm was made in the proximal sternum to allow visualization of the aortic arch. The transverse aortic arch was ligated between the innominate and left common carotid arteries with an overlaid 27-gauge needle. The needle was then immediately removed, leaving a discrete region of constriction.

Echocardiography and In Vivo Hemodynamics.

B6C3F1 mice were acquired from Jackson laboratories. Mice were anesthetized with intraperitoneal ketamine (100 μg/g) for echocardiographic analysis. Two-dimensional images and M-mode tracings were recorded on the short-axis at the level of the papillary muscle to determine percent fractional shortening and ventricular dimensions (GE Vivid□Vision). One day after echocardiography, in vivo hemodynamics was performed using a 1.2 Fr pressure-volume (PV) conductance catheter (Scisense, Ontario, Canada). Mice were anesthetized with an intraperitoneal injection mixture of urethane (1 mg/g), etomidate (10 μg/g), and morphine (1 μg/g) and were then intubated via a tracheotomy and mechanically ventilated at 7 μl/g tidal volume and 120 respirations/min. The PV catheter was placed in the left ventricle via an apical stab approach as previously described (Pacher et al., Nature Protocols 3:1422 (2008). Pressure-volume data were analyzed using IOX2 software (EMKA technologies). All procedures were approved by and performed in accordance with the Institutional Animal Care and Use Committee of the Mount Sinai School of Medicine. The investigation conforms with the Guide for the Care and Use of Laboratory Animals published by the US National Institutes of Health (NIH Publication No. 85-23, revised 1996).

Histological Examination of Cardiac Tissues.

To measure cardiomyocyte cross sectional area, suitable cross-sections with nearly circular capillary profiles and nuclei were selected. These were observed using an Axiophot microscope (Carl Zeiss, Germany), and then analyzed using the Analysis-SIS3.2 software (Soft-Imaging System, Germany). To measure the fibrotic areas, Masson-trichrome staining kit (Sigma-Aldrich (HT15-1kt), USA) was used for staining of the sectioned hearts. The fibrotic areas stained blue and the normal tissue stained red. The fibrotic area was calculated as the ratio of the total area of fibrosis to the total area of the section.

Formulas for miR-25 Use in Cardiomyopathies, Heart Function and Cardiomyocyte Function

Fractional Shortening:

This is determined by echocardiography. There are multiple methods for determining the fraction of contractile movement along a particular axis (short axis) of the left ventricle. A commonly used measurement is:

Left ventricular short axis fractional shortening

FS=[(end-diastolic diameter−end-systolic diameter)/end-diastolic diameter]×100(%)

where
Measurement of LV diameters (mm in the mouse, cm in human):
End-diastolic diameter at Q wave of ECG
End-systolic diameter at either:
a) end-systolic notching of IVS
b) first wide component of II heart sound (phonocardiogram)
c) maximum anterior position of posterolateral wall
Additional formulas are used clinically to measure contractility, some focus on particular parts of the wall.

Ejection Fraction.

There are multiple methods recommended for calculating chamber volumes in the heart using echocardiographic data (Lang et al., J. Amer. Society of Echocardiography 18:1440).

Ejection fraction is the difference between the end diastolic volume and end systolic volume as a fraction of the end diastolic volume:

Ejection fraction(EF)=(EDV−ESV)/EDV

where
EDV is end diastolic volume
ESV is end systolic volume

There are many methods used to determine volume, V, in both end systole and end diastole, most commonly: 1) echocardiography, 2) hemodynamics, 3) magnetic resonance imaging, 4) other imaging methods, e.g. radionuclide ventriculography

For echocardiographic determination, there are also many possibilities, including but not limited to:

$$V = \frac{L}{4} ( A 1 + \frac{A 2 + A 3}{2} + \frac{A 3 + A 4}{2} + \frac{1}{3} A 4 )$$

where A1, A2, A3 and A4 are the Simpson's areas determined by the Simpson's formula automatically by the ultrasound instrument.

Another method is the biplane method of discs (modified Simpson's rule) and is a current method of choice for 2D echocardiography. The principle underlying this method is that the total LV volume is calculated from the summation of a stack of elliptical discs. The height of each disc is calculated as a fraction (usually one-twentieth) of the LV long axis based on the longer of the two lengths from the two- and four-chamber views. This is also done automatically, typically in larger hearts. Its formula is:

$$V = \frac{π}{4} {\sum }_{i = 1}^{20} ai * bi * \frac{L}{20}$$

One skilled in the art will recognize that additional methods are commonly used in clinics to calculate volumes echocardiographically, in particular 3-D real time measurements.

Other methods include but are not limited to calculating directly from the catheter placed into the left ventricle; as well as the use of MRI.

Although the invention has been described with reference to the above examples, it will be understood that modifications and variations are encompassed within the spirit and scope of the invention. Accordingly, the invention is limited only by the following claims.

What is claimed is:

1. A method of increasing contractility of heart muscle or cardiomyocytes in a subject comprising administering a miR-25 inhibitor to the subject, thereby increasing contractility of the heart muscle or cardiomyocytes.

2. The method of claim 1, wherein the miR-25 inhibitor is selected from the group consisting of an antagonist, a peptide, a polynucleotide, an antibody, a polypeptide, a carbohydrate, a small molecule, a peptidomimetic, a siRNA or an antisense oligonucleotide or RNA molecule.

3. The method of claim 2, wherein the miR-25 inhibitor is a miR-25 antagonist.

4. The method of claim 1, wherein the subject has heart failure or cardiomyopathy.

5. The method of claim 1, wherein the levels of proteins regulated by miR-25 are modulated following administration of the miR-25 inhibitor.

6. The method of claim 5, wherein the proteins regulated by miR-25 are selected from the group consisting of Acbd4, Adam23, Fbxw7, Lmbr11, NCK2, NOX4, Plekhm1, Rab8b, SERCA2a, Tmem184b, Txc39b, Whsc111, Wwp2, and zinc and ring finger 2, or any combination thereof.

7. The method of claim 6, wherein the levels of SERCA2a are increased after treatment compared to levels prior to administration of the miR-25 inhibitor.

8. The method of claim 1, wherein the miR-25 inhibitor is administered by oral, transdermal, intravenous, intramuscular, or subcutaneous routes.

9. A method of treating heart failure or cardiomyopathy, comprising administering to a subject a miR-25 inhibitor to the subject, thereby treating heart failure or cardiomyopathy in the subject.

10. A method of increasing SERCA2a levels or function in a subject comprising administration of a miR-25 inhibitor, thereby increasing SERCA2a levels or function in the subject.

11. The method of claim 10, wherein the subject has heart failure or cardiomyopathy.

12. A method of increasing calcium uptake of heart muscle or cardiomyocytes, comprising contacting heart muscle or cardiomyocytes with a miR-25 inhibitor, thereby increasing calcium uptake.

13. The method of claim 12, wherein SERCA2a levels are increased.

14. A pharmaceutical composition comprising a miR-25 inhibitor and a pharmaceutically acceptable carrier.

15. A method of identifying an agent that decreases miR-25 expression or inhibits miR-25 activity comprising:
a) measuring expression levels of miR-25 or SERCA2a in a cell;
b) contacting the cell with a test agent;
c) measuring expression levels of miR-25 or SEARCA2a in the cell; and
d) determining if expression levels of miR-25 or SERCA2a have decreased,
thereby identifying an agent which decreases miR-25 expression or inhibits miR-25 activity.

16. The method of claim 15, wherein the cell is a cardiac cell.

17. The method of claim 16, wherein the cardiac cell is from a subject.

18. The method of claim 15, wherein the test agent is used for treating a subject.

19. A method of diagnosing a cardiac contractile function disorder in a subject comprising comparing the expression level of miR-25 or SERCA2a in a test sample from the subject to the expression level of miR-25 or SERCA2a in a normal sample, wherein a difference in expression level of miR-25 or SERCA2a is diagnostic of a cardiac contractile disorder.

20. A method of monitoring a therapeutic regimen for treating a subject having heart failure by determining a change in expression level of miR-25 or SERCA2a during therapy.

21. A method of improving or treating cardiac function, cardiac output, fractional shortening or fibrosis of heart muscle in a subject comprising administering to the subject an effective amount of an inhibitor of miR-25.


SEQUENCE LISTING (11 sequences)

SEQ ID NO: 1 - DNA, 32 bp, Artificial Sequence
  Primer
cggggtacctgcaatactggagtaaccgcttc

SEQ ID NO: 2 - DNA, 34 bp, Artificial Sequence
  Primer
cggcggccgcatttacctgaaaccatgtctgtgc

SEQ ID NO: 3 - DNA, 40 bp, Artificial Sequence
  Primer
gcagtagacagatgttgttcgaatacaaatattgtgatgc

SEQ ID NO: 4 - DNA, 40 bp, Artificial Sequence
  Primer
gcatcacaatatttgtattcgaacaacatgtgtctactgc

SEQ ID NO: 5 - DNA, 41 bp, Artificial Sequence
  Primer
atgttttttataaaactcatatgtacgaattatgcaatcac

SEQ ID NO: 6 - DNA, 41 bp, Artificial Sequence
  Primer
gtgattgcataattcgtacatatgagttttataaaaaacat

SEQ ID NO: 7 - RNA, 25 nt, Homo sapiens
gcaguagacagauguuggugcaaua

SEQ ID NO: 8 - RNA, 25 nt, Artificial Sequence
  Synthetic construct
gcaguagacagauguuguucgaaua

SEQ ID NO: 9 - RNA, 26 nt, Homo sapiens
auguuuuuuauaaaacugugcaauac

SEQ ID NO: 10 - RNA, 26 nt, Artificial Sequence
  Synthetic construct
auguuuuuuauaaaacucauauguac

SEQ ID NO: 11 - RNA, 22 nt, Homo sapiens
cauugcacuugucucggucuga